(19) United States
(12) Patent Application Publication
(10) Pub. No.: US 2017/0239101 A1
(43) Pub. Date: Aug. 24, 2017

(54) EXTENDABLE ABSORBENT ARTICLE

(71) Applicant: Kimberly-Clark Worldwide, Inc, Neenah, WI (US)

(72) Inventors: KyuLe Lee, Seongnam-si (KR); CaiShan Tan, Seongnam-si (KR); JaeYoung Yoo, Seongnam-si (KR); Franz Aschenbrenner, Kastl (KR)

(21) Appl. No.: 15/506,164

(22) PCT Filed: Aug. 28, 2014
(86) PCT No.: PCT/US2014/053167

§ 371 Date: Feb. 23, 2017

(51) Int. Cl. A61F 13/47; A61F 13/474; A61F 13/56; A61F 13/472

(52) U.S. Cl. CPC A61F 13/4704; A61F 13/472; A61F 13/474; A61F 13/5616; A61F 13/5611; A61F 2013/4708

(57) ABSTRACT

An absorbent article which can be more readily adaptable to meet the needs of the individual wearer of the absorbent article includes a topsheet layer, a backsheet layer, and an absorbent core between the topsheet layer and the backsheet layer. The absorbent article further includes an extendable portion capable of being utilized by the wearer to provide additional area of coverage as deemed suitable by the wearer of the absorbent article.

### BACKGROUND

Products such as absorbent articles are often used to collect and retain human body exudates containing, for example, urine, menses and/or blood. Comfort, absorbency, and discretion are three main product attributes and areas of concern for the wearer of the product. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of feminine pads, sanitary napkins, panty shields, pantiliners, and incontinence devices. These products generally have an absorbent core positioned between a body-facing liquid permeable topsheet layer and a garment-facing liquid impermeable backsheet layer. The edges of the topsheet and the backsheet layers are often bonded together at their periphery to form a seal to contain the absorbent core and body exudates received into the product through the topsheet layer. In use, such products are typically positioned in the crotch portion of an undergarment for absorption of the body exudates and a garment attachment adhesive on the backsheet layer can be used to attach the product to the inner crotch portion of the undergarment. Some of these products can also include wing-like structures for wrapping about the wearer's undergarment to further secure the product to the undergarment and to protect the undergarment from staining. Such wing-like structures (also known as flaps or tabs) are frequently made from lateral extensions of the topsheet and/or backsheet layers.

These commercial products, however, suffer from certain drawbacks. Commercially available products are not always capable of meeting a woman's immediate needs and, therefore, can experience leakage of body exudates from the product. For example, a product may be too small, narrow, wide, or long, may inadequately attach to a woman's undergarment and/or have insufficient capacity for the body exudates during the time period of use. For example, a certain percentage of the female population has an extremely heavy flow during certain portions of the menstrual cycle while during some portions of a menstrual cycle a woman may experience a light flow of menses. Additionally, some women may have heavy menstrual discharge early in the morning due to retention of menses during sleep and a sudden discharge upon awakening. Women may also experience leakage of body exudates from the back of the product, such as when sleeping, which can occur when the body exudates moves backward along the body towards an area where coverage is not being provided by the product. Current commercial products can be deficient in providing the area of coverage that a woman may feel is necessary to provide sufficient protection against leakage and staining.

The variety of activities a wearer may participate in can also create difficulties with the product. For example, the inner crotch portion of an undergarment, to which these products are adhered, is continually distorted, twisted and stretched due to the dynamics of the wearer of the product. As a result, the product may detach from the undergarment causing the undesirable consequence of the product moving out of place. Furthermore, while the product frequently reattaches to the undergarment, the reattachment often places the product in an undesirable position wherein the product will no longer function properly.

Additionally, while attempts have been made to design such products to provide a better contoured fit in the perineal area, one of the difficulties in attempting to design such a product is that women have an almost infinite variety of body shapes and muscle tone in the upper thigh region and, therefore, products which may provide superior comfort and protection for some women due to their configuration, may actually be deficient in these characteristics when worn by women with a different body type.

While feminine care products are widely used, leakage of body exudates remains a top ranked concern among wearers of such products. Such leakage can result in staining of undergarments, outer garments, and/or bedsheets which is unacceptable to the wearer. Conventional products have attempted to address the concern of product leakage through a variety of efforts such as by adding additional absorbent layers within the feminine care product. Conventional products, however, have not provided the desired combination of comfort, versatility and leakage protection. For example, conventional products have not been able to deliver a high level of protection against leakage without the additional discomfort of providing a larger or bulkier feminine care product. A wearer, such as, for example, a woman responding to her level of menstrual flow, may, therefore, resort to utilizing multiple different products throughout the duration of time during which a need exists for an absorbent product.

As a result, there remains a need for an improved product, such as an absorbent article, that is more readily adaptable and which can provide additional coverage as needed by the individual wearer.

### SUMMARY

In various embodiments, an absorbent article can have a longitudinal direction and a transverse direction; a first longitudinal length and a first transverse width; a topsheet layer, a backsheet layer, and an absorbent core positioned between the topsheet layer and the backsheet layer; a first extendable portion having a first portion of the topsheet layer, a first portion of the backsheet layer and a folded configuration; a second extendable portion having a second portion of the topsheet layer, a second portion of the backsheet layer and a folded configuration; and a first configuration capable of converting to a second configuration, wherein the first extendable portion is capable of extending the absorbent article in the longitudinal direction in the conversion of the absorbent article from the first configuration to the second configuration, and wherein the second extendable portion is capable of extending the absorbent article in the transverse direction of the absorbent article. In various embodiments, the first extendable portion further has a portion of the absorbent core between the topsheet layer and the backsheet layer. In various embodiments, the second extendable portion further has a portion of the absorbent core between the topsheet layer and the backsheet layer. In various embodiments, the folded configuration of the first extendable portion further has a garment attachment mechanism. In various embodiments, the folded configuration of the second extendable portion further has a garment attachment mechanism. In various embodiments, the folded region of the first extendable portion is one of a Z-fold or S-fold. In various embodiments, the folded region of the second extendable portion is one of a Z-fold or S-fold.

In various embodiments, an absorbent article has a longitudinal direction and a transverse direction; a first longitudinal length and a first transverse width; a topsheet layer, a backsheet layer, and an absorbent core positioned between the topsheet layer and the backsheet layer; a first extendable portion having a laminate of a liquid permeable layer, a liquid impermeable layer and a folded configuration, wherein at least a portion of the laminate is permanently attached to the backsheet layer; and a first configuration capable of converting to a second configuration, wherein the first extendable portion is capable of extending the absorbent article in one of the longitudinal direction or transverse direction in the conversion of the absorbent article from the first configuration to the second configuration. In various embodiments, a portion of the liquid permeable layer of the first extendable portion is in a face-to-face relationship with the backsheet layer. In various embodiments, the first extendable portion further comprises an absorbent material positioned between the liquid permeable layer and the liquid impermeable layer.

In various embodiments, the absorbent article further has a second extendable portion having a laminate of a liquid permeable layer, a liquid impermeable layer, and a folded configuration, wherein at least a portion of the laminate is permanently attached to the backsheet layer, and wherein the second extendable portion is capable of extending the absorbent article in one of the longitudinal direction or the transverse direction of the absorbent article. In various embodiments, the second extendable portion further has an absorbent material positioned between the liquid permeable layer and the liquid impermeable layer.

In various embodiments, the absorbent article further has a second extendable portion having a first portion of the topsheet layer, a first portion of the backsheet layer and a folded configuration, wherein the second extendable portion is capable of extending the absorbent article in one of the longitudinal direction or the transverse direction of the absorbent article. In various embodiments, the second extendable portion further comprises a portion of the absorbent core between the topsheet layer and the backsheet layer.

In various embodiments, the first extendable portion is capable of extending in the longitudinal direction and the second extendable portion is capable of extending in the transverse direction. In various embodiments, the first extendable portion is capable of extending in the transverse direction and the second extendable portion is capable of extending in the longitudinal direction.

### BRIEF DESCRIPTION OF THE DRAWINGS

FIG. 1A is a top view of an exemplary embodiment of an extendable absorbent article extended in the longitudinal direction of the absorbent article.

FIG. 1B is a top view of the absorbent article of FIG. 1A, in a first configuration and capable of undergoing extension in the longitudinal direction of the absorbent article.

FIG. 1C is a cross-sectional view of the absorbent article of FIG. 1B taken along line 1C-1C.

FIG. 1D is a bottom view of the absorbent article of FIG. 1A.

FIG. 2A is a top view of an exemplary embodiment of an extendable absorbent article extended in the longitudinal direction of the absorbent article.

FIG. 2B is a top view of the absorbent article of FIG. 2A, in a first configuration and capable of undergoing extension in the longitudinal direction of the absorbent article.

FIG. 2C is a cross-sectional view of the absorbent article of FIG. 2B taken along line 2C-2C.

FIG. 2D is a bottom view of the absorbent article of FIG. 2A

FIG. 3A is a top view of an exemplary embodiment of an extendable absorbent article extended in the transverse direction of the absorbent article.

FIG. 3B is a top view of the absorbent article of FIG. 3A, in a first configuration and capable of undergoing extension in the transverse direction of the absorbent article.

FIG. 3C is an exemplary cross-sectional view of an embodiment of the absorbent article of FIG. 3B taken along line 3C-3C.

FIG. 3D is a second exemplary cross-sectional view of an embodiment of the absorbent article of FIG. 3B taken along line 3D-3D.

FIG. 3E is a bottom view of the absorbent article of FIG. 3A.

FIG. 4A is a top view of an exemplary embodiment of an extendable absorbent article extended in the longitudinal direction of the absorbent article and the transverse direction of the absorbent article.

FIG. 4B is a top view of the absorbent article of FIG. 4A, in a first configuration and capable of undergoing extension in the longitudinal direction of the absorbent article and in the transverse direction of the absorbent article.

FIG. 4C is a bottom view of the absorbent article of FIG. 4A.

FIG. 5A is a top view of an exemplary embodiment of an extendable absorbent article extended in the longitudinal direction of the absorbent article and the transverse direction of the absorbent article.

FIG. 5B is a top view of the absorbent article of FIG. 5A, in a first configuration and capable of undergoing extension in the longitudinal direction of the absorbent article and in the transverse direction of the absorbent article.

FIG. 5C is a bottom view of the absorbent article of FIG. 5A.

FIG. 6A is a top view of an exemplary embodiment of an extendable absorbent article extended in the longitudinal direction of the absorbent article.

FIG. 6B is a top view of the absorbent article of FIG. 6A, in a first configuration and capable of undergoing extension in the longitudinal direction of the absorbent article.

FIG. 6C is a cross-sectional view of the absorbent article of FIG. 6B, taken along line 6C-6C.

FIG. 6D is a bottom view of the absorbent article of FIG. 6A.

FIG. 7A is a top view of an exemplary embodiment of an extendable absorbent article extended in the longitudinal direction of the absorbent article.

FIG. 7B is a top view of the absorbent article of FIG. 7A, in a first configuration and capable of undergoing extension in the longitudinal direction of the absorbent article.

FIG. 7C is a cross-sectional view of the absorbent article of FIG. 7B, taken along line 7C-7C.

FIG. 7D is a bottom view of the absorbent article of FIG. 7A.

FIG. 8A is a top view of an exemplary embodiment of an extendable absorbent article extended in the transverse direction of the absorbent article.

FIG. 8B is a top view of the absorbent article of FIG. 8A, in a first configuration and capable of undergoing extension in the transverse direction of the absorbent article.

FIG. 8C is an exemplary cross-sectional view of an embodiment of the absorbent article of FIG. 8B, taken along line 8C-8C.

FIG. 8D is a second exemplary cross-sectional view of an embodiment of the absorbent article of FIG. 8B, taken along line 8D-8D.

FIG. 8E is a bottom view of the absorbent article of FIG. 8A.

FIG. 9A is a top view of an exemplary embodiment of an extendable absorbent article extended in the longitudinal direction of the absorbent article and in the transverse direction of the absorbent article.

FIG. 9B is a top view of the absorbent article of FIG. 9A, in a first configuration and capable of undergoing extension in the longitudinal direction of the absorbent article and in the transverse direction of the absorbent article.

FIG. 9C is a bottom view of the absorbent article of FIG. 9A.

FIG. 10A is a top view of an exemplary embodiment of an extendable absorbent article extended in the longitudinal direction of the absorbent article and in the transverse direction of the absorbent article.

FIG. 10B is a top view of the absorbent article of FIG. 10A, in a first configuration and capable of undergoing extension in the longitudinal direction of the absorbent article and in the transverse direction of the absorbent article.

FIG. 10C is a bottom view of the absorbent article of FIG. 10A.

### DETAILED DESCRIPTION

The present disclosure is generally directed towards an absorbent article, which can be more readily adaptable to meet the needs of the individual wearer of the absorbent article. The absorbent article can have a topsheet layer, a backsheet layer, and an absorbent core between the topsheet layer and the backsheet layer. The absorbent article can further have at least one extendable portion. An extendable portion can be utilized by the wearer to provide additional area of coverage as deemed suitable by the wearer of the absorbent article. In various embodiments, an absorbent article can have at least one extendable portion which can extend the absorbent article in the longitudinal direction of the absorbent article. In various embodiments, an absorbent article can have at least one extendable portion which can extend the absorbent article in the transverse direction of the absorbent article. In various embodiments, an absorbent article can have at least one extendable portion capable of extending the absorbent article in the longitudinal direction of the absorbent article and at least one extendable portion capable of extending the absorbent article in the transverse direction of the absorbent article.

The absorbent article can have a first configuration in which the absorbent article can have a first longitudinal length and a first transverse width. Prior to usage of the absorbent article, and dependent upon their needs which they intend to be addressed by the absorbent article, the wearer of the absorbent article can manipulate the absorbent article, such as by manipulating an extendable portion, and convert the absorbent article from the first configuration to a second configuration. In various embodiments, manipulating an extendable portion can include the step of extending an extendable portion in the longitudinal or transverse direction. In such embodiments, extending an extendable portion can increase at least one of the first longitudinal length or first transverse width to a second longitudinal length or second transverse width, respectively.

In various embodiments, such as, for example, in a situation in which the wearer has no desire or need for additional area of coverage beyond the area of coverage already provided by the first longitudinal length and the first transverse width of an absorbent article in a first configuration, the conversion of the absorbent article from a first configuration to a second configuration can include the step of maintaining an extendable portion in its first configuration (i.e., a step of refraining from extending an extendable portion). The absorbent article, in the second configuration, therefore, can have a second longitudinal length and a second transverse width which, in such embodiments, are the same as the first longitudinal length and the first transverse width of the absorbent article in the first configuration. The absorbent article can, therefore, be utilized by the wearer in the first configuration.

Definitions:

As used herein, the term “absorbent article” refers herein to a garment or other end-use personal care absorbent article, including, but not limited to, catamenial products, such as sanitary napkins, feminine pads, pantiliners, and panty shields, incontinence devices, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber. Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

Absorbent Article:

The present disclosure is generally directed towards a product, such as an absorbent article, which can be more readily adaptable to meet the needs of the individual wearer of the absorbent article. The absorbent article can have a topsheet layer, a backsheet layer, and an absorbent core between the topsheet layer and the backsheet layer. The absorbent article can further have at least one extendable portion. In various embodiments, an absorbent article can have at least one extendable portion which can extend the absorbent article in the longitudinal direction of the absorbent article. In various embodiments, an absorbent article can have at least one extendable portion which can extend the absorbent article in the transverse direction of the absorbent article. An extendable portion can be utilized by the wearer to provide additional area of coverage as deemed suitable by the wearer of the article.

In various embodiments, an absorbent article can have at least one extendable portion which can be constructed as an extension of the topsheet layer, the backsheet layer, and in various embodiments an absorbent material, such as any absorbent material described herein as suitable for an absorbent core or fluid intake layer, such that the extendable portion can be of a unitary construction with the absorbent article. In various embodiments, an absorbent article can have at least one extendable portion which can be constructed of materials similar to the topsheet layer, backsheet layer, and in various embodiments an absorbent material, such as any absorbent material described herein as suitable for use in an absorbent core or fluid intake layer, and at least a portion of such an extendable portion can be permanently bonded with a layer of the absorbent article, such as for example, the backsheet layer of the absorbent article. In various embodiments, an absorbent article can have at least one extendable portion which can be constructed as an extension of the topsheet layer, the backsheet layer, and in various embodiments an absorbent material, such as any absorbent material described herein as suitable for an absorbent core or fluid intake layer, and the absorbent article can have at least one extendable portion which can be constructed of materials similar to the topsheet layer, backsheet layer, and in various embodiments an absorbent material, such as any absorbent material described herein as suitable for use in an absorbent core or fluid intake layer, and at least a portion of such an extendable portion can be permanently bonded with a layer of the absorbent article, such as, for example, the backsheet layer of the absorbent article.

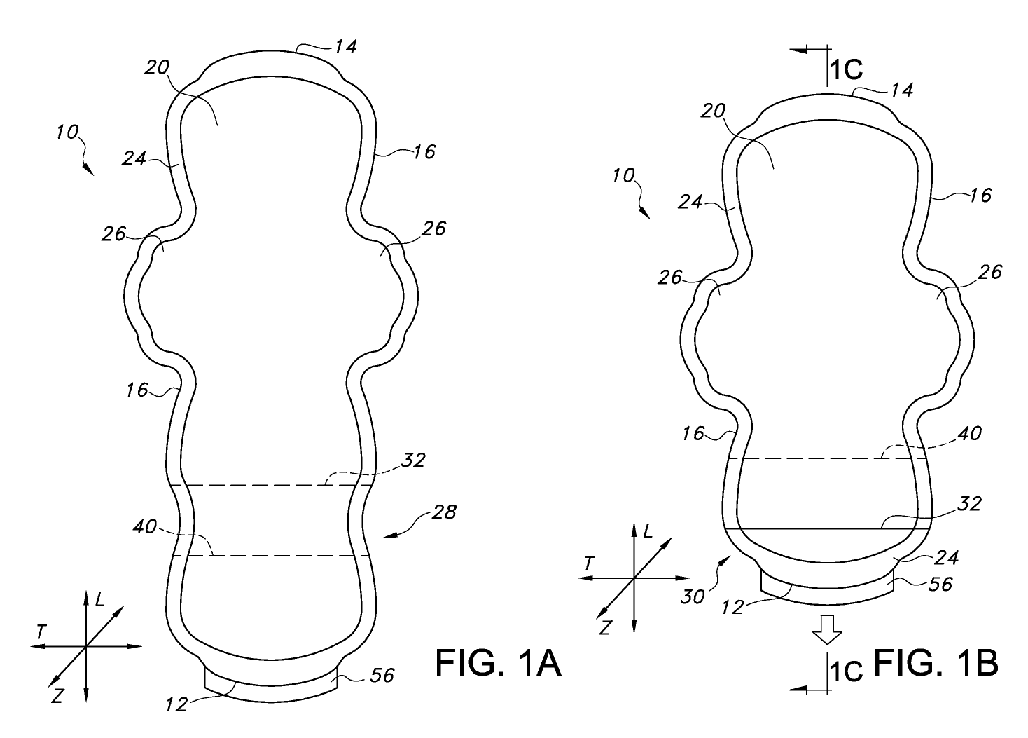

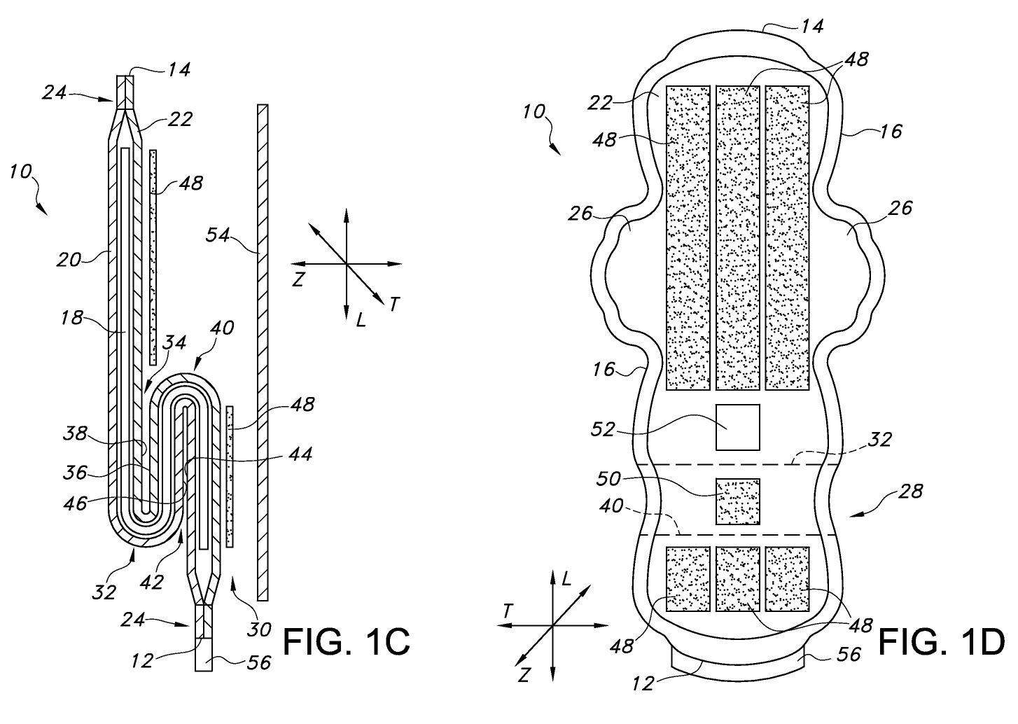

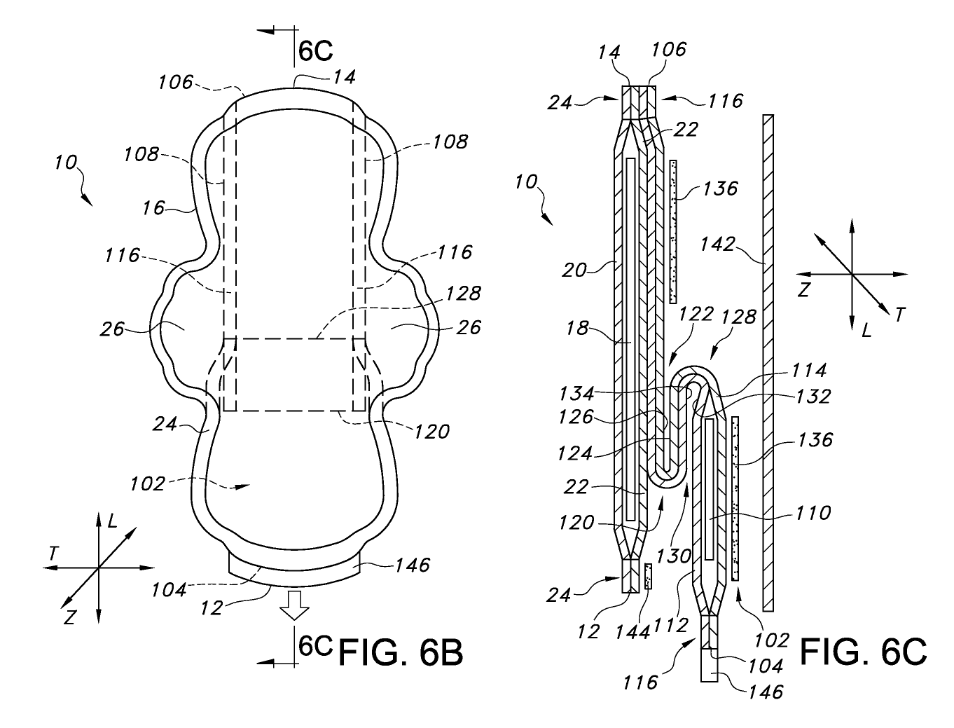

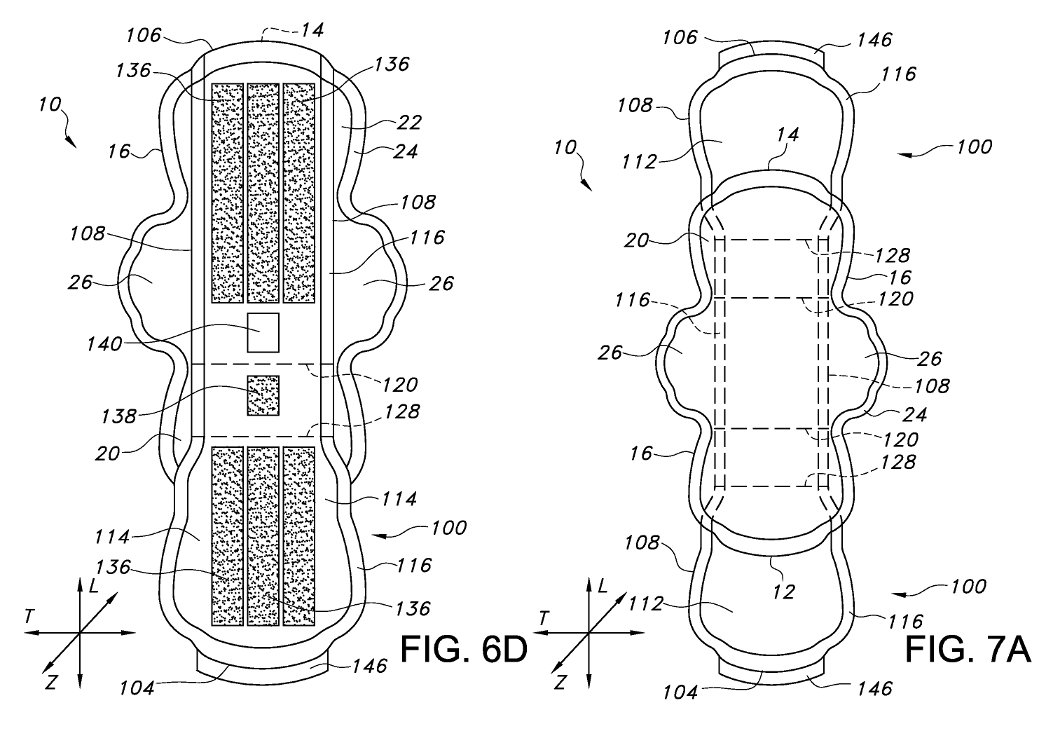

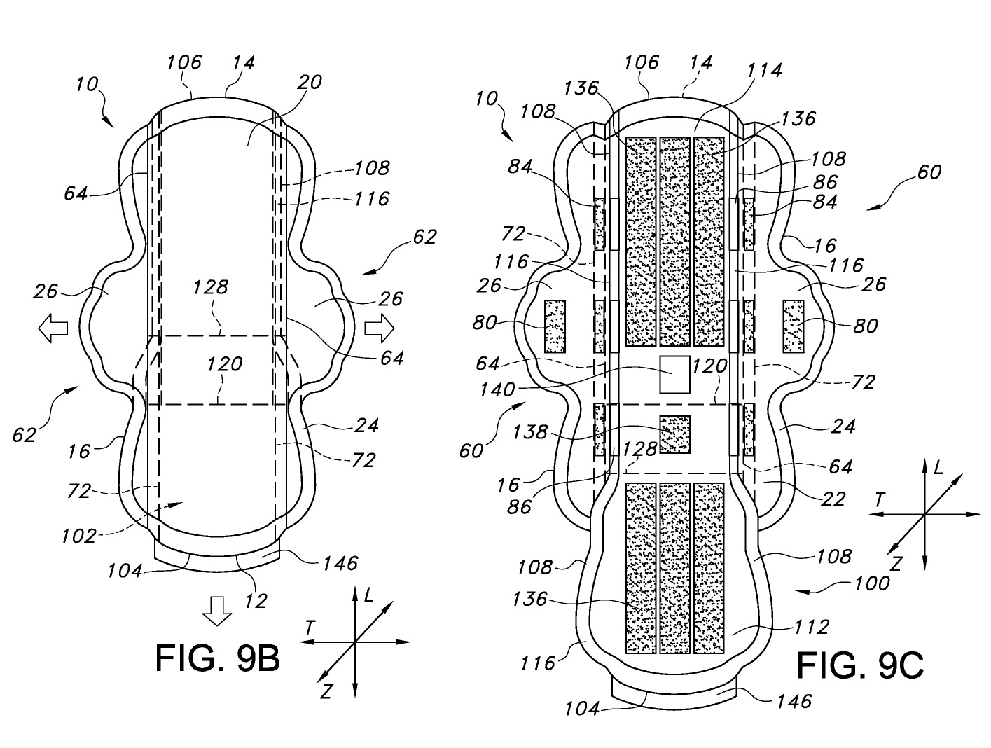

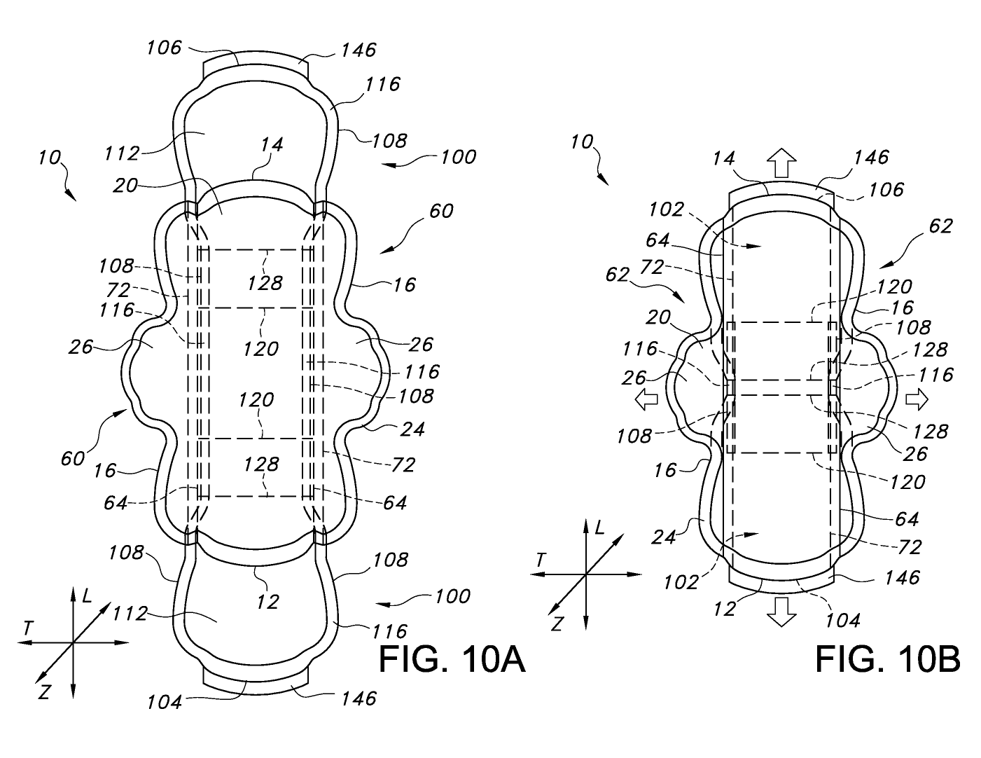

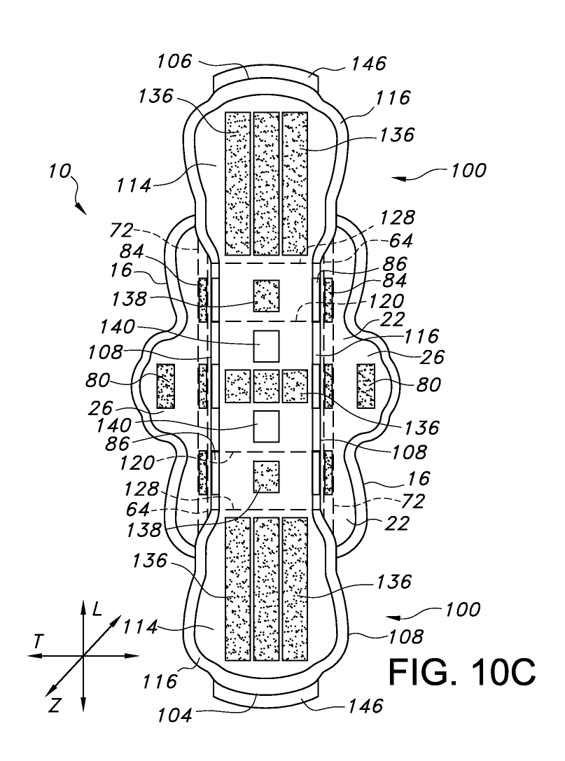

A non-limiting variety of exemplary embodiments of an extendable absorbent article will be described and illustrated herein. It is to be understood that additional embodiments can be constructed utilizing the details described and illustrated herein. FIGS. 1A-1D, 2A-2D, 3A-3E, 4A-4C, and 5A-5C provide various exemplary embodiments of an extendable absorbent article 10 which can have at least one extendable portion which can be constructed as an extension of a topsheet layer 20, a backsheet layer 22, and/or an absorbent material, such as any absorbent material described herein as suitable for use as an absorbent core 18 or fluid intake layer, of an absorbent article 10. FIGS. 6A-6D, 7A-7D and 8A-8E provide various exemplary embodiments of an extendable absorbent article 10 which can have at least one extendable portion which can be constructed of materials similar to the topsheet layer 20, the backsheet layer 22, and/or an absorbent material, such as any material described herein as suitable for an absorbent core 18 or fluid intake layer, and at least a portion of the extendable portion can be permanently bonded with a layer of the absorbent article 10, such as, for example, the backsheet layer 22 of the absorbent article 10. FIGS. 9A-9C and 10A-10C provide various exemplary embodiments of an extendable absorbent article 10 which can have at least one extendable portion constructed as an extension of a topsheet layer 20, a backsheet layer 22, and/or an absorbent material, such as any absorbent material described herein as suitable for the absorbent core 18 or fluid intake layer, and the absorbent article 10 can have at least one extendable portion constructed from materials similar to the topsheet layer 20, the backsheet layer 22, and/or an absorbent material, such as any absorbent material described herein as suitable for the absorbent core 18 or fluid intake layer, and at least a portion of such an extendable portion can be permanently bonded with a layer of the absorbent article 10, such as, for example, the backsheet layer 22 of the absorbent article 10.

Referring to the FIGURES, the absorbent article 10 can have a longitudinal direction (L), a transverse direction (T), and a depth direction (Z). The absorbent article 10 can have a first transverse direction end edge 12, a second transverse direction end edge 14 opposite the first transverse direction end edge 12, and a pair of opposing longitudinal direction side edges 16. The absorbent article 10 can have a garment facing, liquid impermeable backsheet layer 22 and a wearer facing, liquid permeable topsheet layer 20. An absorbent core 18 can be positioned between the backsheet layer 22 and the topsheet layer 20. In various embodiments, the absorbent article 10 can take on various geometries but will generally have a pair of opposing longitudinal direction side edges 16 and a pair of opposing transverse direction end edges 12 and 14. The absorbent article 10 can have a first configuration. In various embodiments, in a first configuration, the absorbent article 10 can have a first longitudinal length which can be the length as measured from the first transverse direction end edge 12 to the second transverse direction end edge 14. In various embodiments, in a first configuration, the absorbent article 10 can have a first transverse width which can be the width as measured from one of the longitudinal direction side edges 16 to the opposing longitudinal direction side edge 16. The first longitudinal length and the first transverse width can be any length and width as deemed suitable.

The topsheet layer 20 and the backsheet layer 22 can both extend beyond the outermost peripheral edges of the absorbent core 18 and can be peripherally bonded together, either entirely or partially, using known bonding techniques to form a sealed peripheral region 24. For example, the topsheet layer 20 and the backsheet layer 22 can be bonded together by adhesive bonding, ultrasonic bonding, or any other suitable bonding method known in the art.

In various embodiments, the absorbent article 10 can have a pair of wings 26 extending outwardly, in the transverse direction T, from the absorbent article 10. The wings 26 can drape over the edges of the wearer's undergarment so that the wings 26 are disposed between the edges of the wearer's undergarment and her thighs. The wings 26 can serve at least two purposes. First, the wings 26 can prevent soiling of the wearer's undergarment by forming a barrier along the edges of the undergarment. Second, the wings 26 can be provided with an attachment aid, such as, for example, a garment attachment adhesive or a hook, to keep the absorbent article 10 securely and properly positioned in the undergarment. The wings 26 can wrap around the crotch region of the wearer's undergarment to aid in securing the absorbent article 10 to the wearer's undergarment when in use. Each wing 26 can fold under the crotch region of the wearer's undergarment and the attachment aid can either form a secure attachment to the opposite wing 26 or directly to the surface of the wearer's undergarment. In various embodiments, the wings 26 can be an extension of materials forming the topsheet layer 20 and/or the backsheet layer 22, such that the wings 26 can be of a unitary construction with the absorbent article 10. In various embodiments, the wings 26 can be constructed of materials similar to the topsheet layer 20, the backsheet layer 22 or combinations of these materials. In various embodiments, the wings 26 can be separate elements bonded to the main body of the absorbent article 10. It is to be understood that the wings 26 are optional and, in various embodiments, an absorbent article 10 can be configured without wings 26.

The absorbent article 10 can have at least one extendable portion. Various embodiments of an extendable portion are described and illustrated herein. In various embodiments, an extendable portion can allow a wearer of the absorbent article 10 to extend the absorbent article 10 in the longitudinal direction L of the absorbent article 10 as desired, forming an extended region. In various embodiments, an extendable portion can allow a wearer of the absorbent article 10 to extend the absorbent article 10 in the transverse direction T of the absorbent article 10, as desired, forming an extended region. In various embodiments, an extendable portion can be an extension of the materials forming the topsheet layer 20, the backsheet layer 22, and in various embodiments an absorbent material, such as, for example, the absorbent core 18, such that the extendable portion can be a unitary construction with the absorbent article 10. In such an exemplary embodiment, the extendable portion can be a portion of the absorbent article 10 which can be folded back under itself. In various embodiments, an extendable portion can be constructed of materials similar to the topsheet layer 20, backsheet layer 22, and in various embodiments an absorbent material such as described herein as suitable for use as an absorbent core 18 or fluid intake layer, and at least a portion of such an extendable portion can be permanently bonded with a layer of the absorbent article 10, such as, for example, the backsheet layer 22 of the absorbent article 10.

Each of these components of the absorbent article 10, as well as additional components and additional exemplary embodiments of the absorbent article 10, will be described in more detail herein.

Topsheet Layer:

The topsheet layer 20 defines a wearer facing surface of the absorbent article 10 that may directly contact the body of the wearer and is liquid permeable to receive body exudates. The topsheet layer 20 is desirably provided for comfort and conformability and functions to direct body exudates away from the body of the wearer, through its own structure, and towards the absorbent core 18. The topsheet layer 20 desirably retains little to no liquid in its structure, so that it provides a relatively comfortable and non-irritating surface next to the tissues within the vestibule of a female wearer.

The topsheet layer 20 can be a single layer of material, or alternatively, can be multiple layers that have been laminated together. The topsheet layer 20 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 20.

In various embodiments, the topsheet layer 20 can be constructed from various nonwoven webs such as meltblown webs, spunbond webs, hydroentangled spunlace webs, or through air bonded carded webs. Examples of suitable topsheet layer 20 materials can include, but are not limited to, natural fiber webs (such as cotton), rayon, bonded carded webs of polyester, polypropylene, polyethylene, nylon, or other heat-bondable fibers, polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 20 can be a bonded carded web made of polypropylene and polyethylene such as that obtainable from Sandler Corporation, Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 20, each of which is hereby incorporated by reference thereto in its entirety. Additional topsheet layer 20 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 20 may contain a plurality of apertures (not shown) formed therethrough to permit body exudates to pass more readily into the absorbent core 18. The apertures may be randomly or uniformly arranged throughout the topsheet layer 20. The size, shape, diameter, and number of apertures may be varied to suit an absorbent article's 10 particular needs.

In various embodiments, the topsheet layer 20 can have a basis weight ranging from about 5, 10, 15, 20 or 25 gsm to about 50, 100, 120, 125 or 150 gsm. For example, in an embodiment, a topsheet layer 20 can be constructed from a through air bonded carded web having a basis weight ranging from about 15 gsm to about 100 gsm. In another example, a topsheet layer 20 can be constructed from a through air bonded carded web having a basis weight from about 20 gsm to about 50 gsm, such as a through air bonded carded web that is readily available from nonwoven material manufacturers, such as Xiamen Yanjan Industry, Beijing DaYuan Nonwoven Fabrics and others.

In various embodiments, the topsheet layer 20 can be at least partially hydrophilic. In various embodiments, a portion of the topsheet layer 20 can be hydrophilic and a portion of the topsheet layer 20 can be hydrophobic. In various embodiments, the portions of the topsheet layer 20 which can be hydrophobic can be either an inherently hydrophobic material or can be a material treated with a hydrophobic coating.

In various embodiments, the topsheet layer 20 can be a multicomponent topsheet layer 20 such as by having two or more different nonwoven or film materials, with the different materials placed in separate locations in the transverse direction T of the absorbent article 10. For example, the topsheet layer 20 can be a two layer or multicomponent material having a central portion positioned along and straddling the longitudinal center direction of the absorbent article 10, with lateral side portions flanking and bonded to each side edge of the central portion. The central portion can be constructed from a first material and the side portions can be constructed from a material which can be the same as or different from the material of the central portion. In such embodiments, the central portion may be at least partially hydrophilic and the side portions may be inherently hydrophobic or may be treated with a hydrophobic coating. Examples of constructions of multi-component topsheet layers 20 are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

Backsheet Layer:

The backsheet layer 22 is generally liquid impermeable and is the portion of the absorbent article 10 which faces the garment of the wearer. The backsheet layer 22 can permit the passage of air or vapor out of the absorbent article 10 while still blocking the passage of liquids. Any liquid impermeable material may generally be utilized to form the backsheet layer 22. The backsheet layer 22 can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the backsheet layer 22 can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a backsheet layer 22 can be a polyethylene film such as that obtainable from Pliant Corporation, Schaumburg, Ill., USA. Another example can include calcium carbonate-filled polypropylene film. In still another embodiment, the backsheet layer 22 can be a hydrophobic nonwoven material with water barrier properties such as a nonwoven laminate, an example of which can be a spunbond, meltblown, meltblown, spunbond, four-layered laminate. The backsheet layer 22 can, therefore, be of a single or multiple layer construction, such as of multiple film layers or laminates of film and nonwoven fibrous layers. Suitable backsheet layers 22 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

Absorbent Core:

An absorbent core 18 can be positioned between the topsheet layer 20 and the backsheet layer 22. The absorbent core 18 can generally be any single layer structure or combination of layer components, which can demonstrate some level of compressibility, conformability, be non-irritating to a wearer's skin, and capable of absorbing and retaining liquids and other body exudates. Additionally, the absorbent core 18 can provide additional capacity to absorb and retain body exudates such as menses. In various embodiments, the absorbent core 18 can be formed from a variety of different materials and can contain any number of desired layers. For example, the absorbent core 18 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers.

In various embodiments, if desired, the absorbent core 18 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 18 in any amount as desired.

Regardless of the combination of absorbent materials used in the absorbent core 18, the absorbent materials can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

The shape of the absorbent core 18 can vary as desired and can comprise any one of various shapes including, but not limited to, triangular, rectangular, dog-bone and elliptical shapes. In various embodiments, the absorbent core 18 can have a shape that generally corresponds with the overall shape of the absorbent article 10. The dimensions of the absorbent core 18 can be substantially similar to those of the absorbent article 10, however, it will be appreciated that the dimensions of the absorbent core 18 while similar, will often be less than those of the overall absorbent article 10, in order to be adequately contained therein.

By way of example, suitable materials and/or structures for the absorbent core 18 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al., each of which is hereby incorporated by reference thereto in its entirety.

Extendable Portion:

In various embodiments, the absorbent article 10 can have at least one extendable portion. In various embodiments, the absorbent article 10 can have at least 1, 2, 3 or 4 extendable portions. An extendable portion can be utilized by a wearer to provide additional area of coverage as deemed suitable by the wearer of the absorbent article 10.

In various embodiments, an absorbent article 10 can have at least one extendable portion which can provide extension of the absorbent article 10 in the longitudinal direction of the absorbent article 10. In various embodiments, an absorbent article 10 can have at least one extendable portion which can provide extension of the absorbent article 10 in the transverse direction of the absorbent article 10.

In various embodiments, an absorbent article 10 can have at least two extendable portions which can each provide extension of the absorbent article 10 in the longitudinal direction of the absorbent article 10. In various embodiments, an absorbent article 10 can have at least two extendable portions which can each provide extension of the absorbent article 10 in the transverse direction of the absorbent article 10. In various embodiments, an absorbent article 10 can have at least two extendable portions wherein at least one of the extendable portions can provide extension of the absorbent article 10 in the transverse direction of the absorbent article 10 and at least one of the extendable portions can provide extension of the absorbent article 10 in the longitudinal direction of the absorbent article 10.

In various embodiments, an absorbent article 10 can have at least three extendable portions wherein two of the extendable portions can provide extension of the absorbent article 10 in the longitudinal direction of the absorbent article 10 and one of the extendable portions can provide extension of the absorbent article 10 in the transverse direction of the absorbent article 10. In various embodiments, an absorbent article 10 can have at least three extendable portions wherein two of the extendable portions can provide extension of the absorbent article 10 in the transverse direction of the absorbent article 10 and one of the extendable portions can provide extension of the absorbent article 10 in the longitudinal direction of the absorbent article 10.

In various embodiments, an absorbent article 10 can have at least four extendable portions wherein two of the extendable portions can provide extension of the absorbent article 10 in the longitudinal direction of the absorbent article 10 and two of the extendable portions can provide extension of the absorbent article 10 in the transverse direction of the absorbent article 10.

In embodiments in which the absorbent article 10 can have more than one extendable portion, each extendable portion can be independently capable of extending the absorbent article 10. As each extendable portion can be independently capable of extending the absorbent article 10, a wearer of the absorbent article 10 can select to extend any one of the extendable portions as desired. A wearer can select to extend one of, all of, or any number in-between of the extendable portions present in the absorbent article 10.

In various embodiments in which an extendable portion can be constructed as an extension of the materials forming the topsheet layer 20 and the backsheet layer 22, the manufacture of the absorbent article 10 can include a step of forming the absorbent article 10 into a fully extended configuration having the fully extended size dimensions. In such a configuration, the topsheet layer 20 and the backsheet layer 22 can both extend beyond the outermost peripheral edges of the absorbent core 18 and can extend to the fully extended size dimension of a fully extended absorbent article 10. The topsheet layer 20 and backsheet layer 22 can be bonded together at their periphery, either entirely or partially, using known bonding techniques to form a sealed peripheral region 24. A portion of the fully extended topsheet layer 20 and backsheet layer 22 can then be folded back and under the remainder of the absorbent article 10. The folding of a portion of the topsheet layer 20 and the backsheet layer 22 can initiate the placement of the absorbent article 10 into a first configuration. The folded portion of the absorbent article 10 can form an extendable portion which can later be manipulated by the wearer of the absorbent article 10 as desired based on the wearer's need. In such embodiments, the topsheet layer 20 and the backsheet layer 22 can extend beyond the outermost peripheral edges of the absorbent core 18 any amount deemed suitable to ultimately provide an extendable portion that can allow for extension of the absorbent article 10 as needed by a wearer of the absorbent article 10. If needed by the wearer, the wearer can manipulate an extendable portion (e.g., unfolding the extendable portion) to extend the dimensions of the absorbent article 10 as desired. If an extendable portion is not needed by the wearer during use, the extendable portion can remain in the folded, first configuration.

In various embodiments in which an extendable portion can be constructed as an extension of the materials forming the topsheet layer 20, the backsheet layer 22, and an absorbent material, the manufacture of the absorbent article 10 can be similar to the manufacture just described above for the manufacture of an absorbent article 10 with an extendable portion constructed as an extension of the materials forming the topsheet layer 20 and the backsheet layer 22. In such a configuration, the topsheet layer 20, the backsheet layer 22, and the absorbent material can all be sized to the fully extended size dimension of a fully extended absorbent article 10. It is to be understood that the topsheet layer 20 and the backsheet layer 22 can still extend beyond the outermost periphery of the absorbent material in order to be bonded together to form a sealed peripheral region 24.

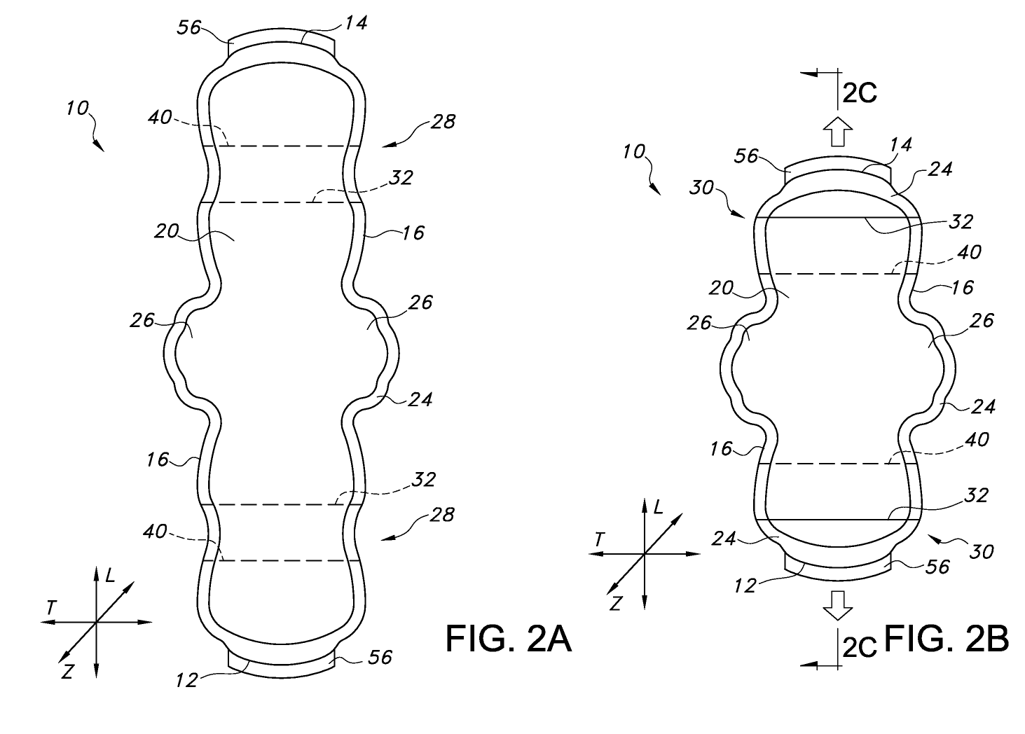

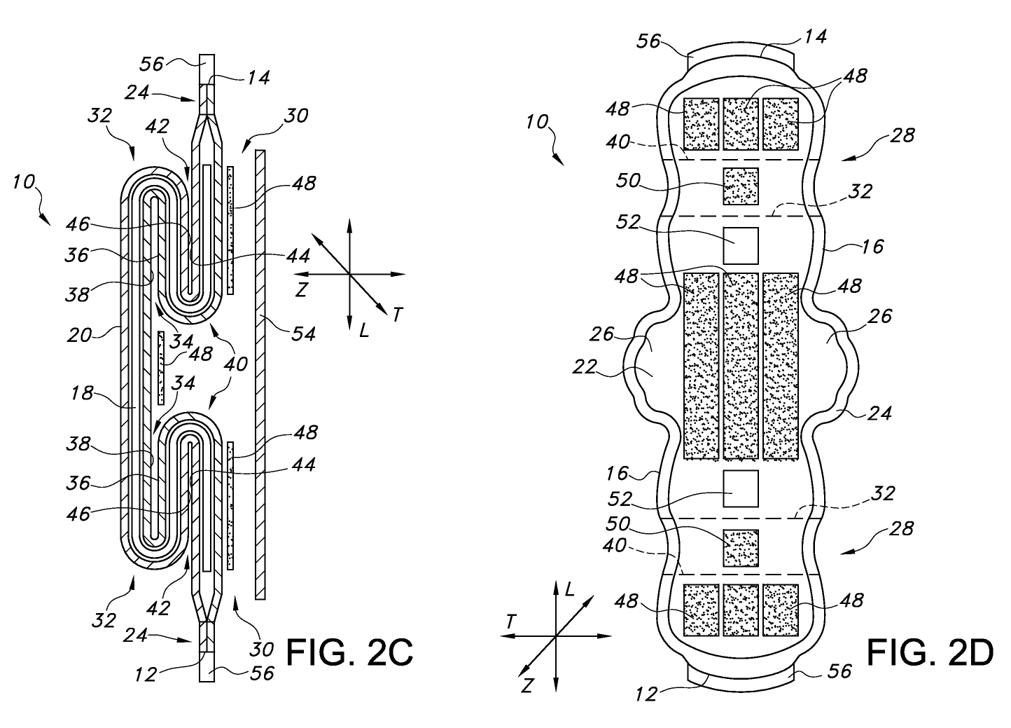

FIGS. 1A-1D and FIGS. 2A-2D provide illustrations of various views of exemplary embodiments of extendable absorbent articles 10 having an extendable portion 30 which can extend the absorbent article 10 in the longitudinal direction of the absorbent article 10. FIGS. 1A-1D provide exemplary illustrations of various views of an exemplary absorbent article 10 having one extendable portion 30. In the embodiment illustrated in FIGS. 1A-1D, the extendable portion 30 is illustrated as being constructed as an extension of the topsheet layer 20, the backsheet layer 22, and the absorbent core 18. FIGS. 2A-2D provide exemplary illustrations of various views of an exemplary absorbent article 10 having two extendable portions 30. In the exemplary embodiment of the absorbent article 10 illustrated in FIGS. 2A-2D, the two extendable portions 30 are mirror images of each other and are each capable of extending the absorbent article 10 in a longitudinal direction of the absorbent article 10. As the two extendable portions 30 are mirror images of each other, they can extend the absorbent article 10 in opposing longitudinal directions of the absorbent article 10. The absorbent article 10 illustrated in FIGS. 2A-2D is similar to the absorbent article illustrated in FIGS. 1A-1D in that each extendable portion of the absorbent article 10 illustrated in FIGS. 2A-2D is illustrated as being constructed as an extension of the topsheet layer 20, the backsheet layer 22, and the absorbent core 18. As described herein, an extendable portion 30 need not include in its construction any absorbent material. Thus, while the absorbent article 10 of FIGS. 1A-1D and the absorbent article 10 of FIGS. 2A-2D are illustrated as having at least one extendable portion including an absorbent core 18 within the construction of the extendable portion 30, an absorbent article 10 can be manufactured similar to those of FIGS. 1A-1D and 2A-2D without an absorbent material, such as the absorbent core 18, within the construction of the extendable portion 30.

Referring to FIGS. 1A-1D and 2A-2D, the absorbent article 10 can have a topsheet layer 20, a backsheet layer 22, an absorbent core 18, a pair of opposing transverse direction end edges, 12 and 14, a pair of opposing longitudinal direction side edges 16, and a sealed peripheral region 24 formed by bonding the peripheral edges of the topsheet layer 20 and the backsheet layer 22 together. The absorbent article 10 illustrated in FIGS. 1A-1D can have one extendable portion 30. The extendable portion 30 and, ultimately the extended region 28 illustrated in FIGS. 1A-1D, can be located at either longitudinal end of the absorbent article 10. The extendable portion 30 can include at least a portion of one of the transverse direction end edges, 12 or 14, of the absorbent article. In the embodiment illustrated in FIGS. 1A-1D, the extendable portion 30 and, ultimately, the extended region 28, can include at least a portion of transverse direction end edge 12. The extendable portion 30 can also include a portion of the sealed peripheral region 24. Referring to FIGS. 2A-2D, the absorbent article 10 illustrated can have two extendable portions 30. Each extendable portion 30 and, ultimately the extended regions 28, illustrated in FIGS. 2A-2D can be located one at each longitudinal end of the absorbent article 10. Each extendable portion 30 can include at least a portion of one of the transverse direction end edges, 12 and 14, of the absorbent article 10. In the embodiment illustrated in FIGS. 2A-2D, one of the extendable portions 30 and, ultimately one of the extended regions 28, can include at least a portion of transverse direction end edge 12 and the other of the extendable portions 30 and, ultimately the other extended region 28, can include at least a portion of transverse direction end edge 14. Each extendable portion 30 can also include a portion of the sealed peripheral region 24.

Prior to usage of the absorbent article 10 by a wearer, the absorbent article 10 can be in a first configuration, such as illustrated in FIGS. 1B, 1C, 2B and 2C, and can have an extendable portion 30 (FIGS. 1B and 1C) or an opposing pair of extendable potions 30 (FIGS. 2B and 2C) ready for use by a wearer to extend the absorbent article 10 if so desired. Each extendable portion 30 can be capable of extending the absorbent article 10 in the longitudinal direction of the absorbent article 10, thereby increasing the first configuration first longitudinal length of the absorbent article 10 to a second configuration second longitudinal length. The two extendable portions 30 illustrated in FIGS. 2B and 2C are mirror images of each other and are each independently capable of extending the absorbent article 10 in the longitudinal direction of the absorbent article 10, thereby, increasing the first configuration first longitudinal length of the absorbent article 10 to a second configuration second longitudinal length.

As described herein, each extendable portion 30 of the absorbent article 10 illustrated in FIGS. 1A-1D and 2A-2D can be incorporated into the absorbent article 10 by forming the absorbent article 10 into a fully extended configuration having the fully extended size dimensions. A portion of the fully extended topsheet layer 20, backsheet layer 22, and absorbent core 18 can then be folded back and under the remainder of the absorbent article 10. The portion of the fully extended absorbent article 10 which can be folded can be the portion of the fully extended absorbent article 10 forming each extended region 28. Incorporating a first fold into an extended region 28 can bring a portion of the extended region 28 back and under a remaining portion of the absorbent article 10. FIGS. 1A and 2A provide exemplary illustrations of a potential location where a first fold, such as fold 32, can be incorporated into the extended region 28 of the absorbent article 10. The absorbent article 10 of FIGS. 1A-1D and 2A-2D can be capable of extending in the longitudinal direction of the absorbent article 10 and, therefore, the fold, such as fold 32, can extend from a first of the longitudinal direction side edges 16 to the second of the longitudinal direction side edges 16. As a result of incorporating first fold 32 into the absorbent article 10, a first region 34 (illustrated in FIGS. 1C and 2C) can be formed wherein a first portion 36 of the backsheet layer 22 can be brought into a face-to-face relationship with a second portion 38 of the backsheet layer 22.

The incorporation of the first fold 32 into the absorbent article 10 can re-position one of the transverse direction end edges, 12 or 14, to be closer in proximity to the opposite transverse direction end edge, 14 or 12, respectively, of the absorbent article 10. Additionally, the first fold 32 can, in essence, become a new transverse direction end edge for the absorbent article 10. A wearer of the absorbent article 10, however, need not extend an extendable portion 30 if they do not have a need for such an extension at the time of usage of the absorbent article 10. In such embodiments when a wearer does not extend the extendable portion 30 and wherein the first fold 32 is the transverse direction end edge of the absorbent article 10, the absorbent article 10 could experience leakage of body exudates as the absorbent article 10 would lack a sealed peripheral region 24 in the area of first fold 32. To provide the absorbent article 10 with an extendable portion 30 and a sealed peripheral region 24 at the perimeter of the absorbent article 10 in a first configuration, a second fold 40 can be provided in order to re-direct a portion of the extendable portion 30 such that a portion of the extendable portion 30 extends beyond the first fold 32. As the extendable portion 30 contains a portion of the sealed peripheral region 24 of the absorbent article 10, a portion of the sealed peripheral region 24 can also now be positioned beyond the first fold 32 with the incorporation of the second fold 40. The absorbent article 10 can have, therefore, a sealed peripheral region 24 along the complete perimeter of the absorbent article 10 in a first configuration (as is visible in FIGS. 1B and 2B). As a result of incorporating the second fold 40 in the absorbent article 10, a second region 42 can be formed wherein a first portion 44 of the topsheet layer 20 can be brought into a face-to-face relationship with a second portion 46 of the topsheet layer 20 (such as seen in FIGS. 1C and 2C). The two folds, 32 and 40, can create a Z-shaped or S-shaped fold pattern in the extendable portion 30.

As illustrated in FIGS. 1C, 1D, 2C and 2D, the backsheet layer 22 of the absorbent article 10 can be provided with a garment attachment mechanism 48. The garment attachment mechanism 48 can be any operative, conventional attachment mechanism, examples of which can include, but are not limited to, adhesives, cohesives, inter-engaging mechanical fastener systems or the like, as well as combinations thereof. For example, in various embodiments, the garment attachment mechanism 48 can include a garment attachment adhesive. Examples of suitable garment attachment adhesives and patterns include, but are not limited to, those described in DE700225U1, U.S. Pat. No. 3,881,490 to Whitehead, et al., U.S. Pat. No. 3,913,580 to Ginocchio, U.S. Pat. No. 4,337,772 to Roeder, et al., GB1349962, and U.S. Publication No. 2007/0073255 to Thomas, et al., each of which are hereby incorporated by reference thereto in its entirety. While a pattern of lines of a garment attachment mechanism 48 is illustrated in FIGS. 1D and 2D, the garment attachment mechanism 48 can be provided on the backsheet layer 22 in any location and in any amount as deemed suitable to enable attachment between the absorbent article 10 and the wearer's undergarment. In various embodiments, the absorbent article 10 can utilize a peel strip layer 54 over a garment attachment mechanism 48 to protect the garment attachment mechanism 48 prior to usage of the absorbent article 10.

Referring to FIGS. 1C, 1D, 2C, and 2D, in various embodiments, while the backsheet layer 22 of the absorbent article 10 can be provided with a garment attachment mechanism 48, the first portion 36 of the backsheet layer 22 and the second portion 38 of the backsheet layer 22 which form the first region 34 in the first configuration of the absorbent article 10 can remain free of a garment attachment mechanism. In various embodiments, a portion of at least one of the first portion 36 of the backsheet layer 22 or the second portion 38 of the backsheet layer 22 can be provided with a first region garment attachment mechanism 50. To provide for engagement between the first portion 36 of the backsheet layer 22 and the second portion 38 of the backsheet layer 22 in the first region 34, the first portion 36 of the backsheet layer 22 can be situated over and engaged with the second portion 38 of the backsheet layer 22 via the first region garment attachment mechanism 50 located within the first region 34. Engagement between the first portion 36 of the backsheet layer 22 and the second portion 38 of the backsheet layer 22 can provide for an extendable portion 30 which can remain in a first configuration. When a wearer of the absorbent article 10 desires to extend the extendable portion 30, the first portion 36 of the backsheet layer 22 can be dis-engaged from the second portion 38 of the backsheet layer 22 in a conversion of the absorbent article 10 from a first configuration to a second configuration. A first region garment attachment mechanism 50 can also help secure the extended region 28 of the absorbent article 10 to a wearer's undergarment in the event that the wearer has extended the extendable portion 30 of the absorbent article 10.

In various embodiments in which the first portion 36 of the backsheet layer 22 is engaged with a second portion 38 of the backsheet layer 22 via a first region garment attachment mechanism 50, it can be beneficial to dis-engage the first portion 36 of the backsheet layer 22 from the second portion 38 of the backsheet layer 22 in order to allow for extension of the extendable portion 30 as desired by the wearer of the absorbent article 10. To provide for such releasable engagement between the first portion 36 of the backsheet layer 22 and the second portion 38 of the backsheet layer 22, a portion of the first portion 36 of the backsheet layer 22 and/or a portion of the second portion 38 of the backsheet layer 22 which would be positioned opposite the first region garment attachment mechanism 50 can be provided with a corresponding first region release component 52. For example, in various embodiments, at least a portion of the first portion 36 of the backsheet layer 22 can be provided with a first region garment attachment mechanism 50 in the form of a garment attachment adhesive and at least a portion of the second portion 38 of the backsheet layer 22 can be coated with a silicone treatment, as the first region release component 52, in a location of the second portion 38 of the backsheet layer 22 which will be opposite the first region garment attachment mechanism 50 positioned on the first portion 36 of the backsheet layer 22. When the first portion 36 of the backsheet layer 22 and the second portion 38 of the backsheet layer 22 are in a first configuration such as illustrated in FIGS. 1C and 2C, the first region release component 52, such as the silicone treatment, is in a face-to-face relationship with the first region garment attachment mechanism 50, such as the garment attachment adhesive. In such embodiments, the silicone treatment which is in the face-to-face relationship with the garment attachment adhesive can prevent a permanent engagement between the first portion 36 of the backsheet layer 22 and the second portion 38 of the backsheet layer 22. The first portion 36 of the backsheet layer 22, therefore, can be capable of dis-engaging from the second portion 38 of the backsheet layer 22 when the wearer of the absorbent article 10 desires to extend the extendable portion 30.

Referring to FIGS. 1D and 2D, a bottom view of an exemplary absorbent article 10 extended in a fully extended configuration is illustrated. FIGS. 1D and 2D provide illustrations of potential locations where folds, such as folds 32 and 40, can be incorporated into the absorbent article 10. In various embodiments, the fully extended configuration of an absorbent article 10 can exist such as, for example, during the manufacturing process prior to folding the absorbent article 10 into a first configuration and prior to incorporating folds, such as folds 32 and 40, into the absorbent article 10. In various embodiments, the fully extended configuration can exist, such as, for example, during usage when a wearer has altered the configuration of the absorbent article 10 from the first configuration to a second configuration by extending the extendable portion(s) 30 into extended region(s) 28.

As illustrated in FIGS. 1D and 2D, a portion of the backsheet layer 22 which will form a first portion 36 of the backsheet layer 22 in a first region 34 when the absorbent article 10 is in a first configuration can be provided with a first region garment attachment mechanism 50 and a portion of the backsheet layer 22 which will form a second portion 38 of the backsheet layer 22 in a first region 34 when the absorbent article 10 is in a first configuration can be provided with a corresponding first region release component 52. When the absorbent article 10 is in a first configuration, the first region garment attachment mechanism 50 and the first region release component 52 can align with each other and can be in a face-to-face relationship. Such alignment can prevent a permanent attachment between the first portion 36 and the second portion 38 of the backsheet layer 22. While the first portion 36 has been illustrated with a first region garment attachment mechanism 50 and the second portion 38 has been illustrated with a first region release component 52, it is to be understood that each of the first region garment attachment mechanism 50 and the first region release component 52 can be located on either or both of the first portion 36 and/or second portion 38 of the backsheet layer 22 in the first region 34.

In various embodiments, each of the first portion 36 and the second portion 38 of the backsheet layer 22 can be provided with a garment attachment mechanism. In such embodiments in which both the first portion 36 and the second portion 38 of the backsheet layer 22 have a garment attachment mechanism, each of the first portion 36 and second portion 38 of the backsheet layer can have a corresponding release component, positioned opposite a garment attachment mechanism on the opposing portion of the backsheet layer 22, which can allow for the garment attachment mechanisms to dis-engage and allow for the unfolding of the extendable portion 30. Therefore, a release component, positioned on the second portion 38 of the backsheet layer 22 can allow for a garment attachment mechanism located on the first portion 36 of the backsheet layer 22 to dis-engage. Similarly, a release component positioned on the first portion 36 of the backsheet layer 22 can allow for a garment attachment mechanism located on the second portion 38 of the backsheet layer 22 to dis-engage. For example, in various embodiments, the first portion 36 of the backsheet layer 22 can be provided with a garment attachment mechanism in the form of a garment attachment adhesive and the second portion 38 of the backsheet layer 22 can be provided with a garment attachment mechanism in the form of a garment attachment adhesive. In such embodiments, each of the first portion 36 of the backsheet layer 22 and the second portion 38 of the backsheet layer 22 can also be at least partially coated with a silicone treatment as the corresponding release component. In such embodiments, the silicone treatment on the first portion 36 of the backsheet layer 22 can align in a face-to-face relationship with a garment attachment adhesive on the second portion 38 of the backsheet layer 22 and a silicone treatment on the second portion 38 of the backsheet layer 22 can align in a face-to-face relationship with an garment attachment adhesive on the first portion 36 of the backsheet layer 22. Such alignment can prevent a permanent attachment between the two portions, first portion 36 and second portion 38, of the backsheet layer 22.

In various embodiments, the first region garment attachment mechanism 50 and the corresponding first region release component 52 can be provided in a pattern arrangement. In various embodiments, the first region garment attachment mechanism 50 can be provided in any pattern as deemed suitable. Exemplary patterns can include, but are not limited to, lines, swirls, dots, circles, ovals, and diamonds. In such embodiments, the corresponding first region release component 52 can be provided in a pattern arrangement which can mirror the pattern of the first region garment attachment mechanism 50.

To assist the wearer with extending the extendable portion 30, the extendable portion 30 can be provided with a tab 56. The tab 56 can be an integral unitary construction with the extendable portion 30 or the tab 56 can be a separate element bonded to the absorbent article 10. The tab 56 can be formed from materials similar to the topsheet layer 20, the backsheet layer 22 or combinations of these materials. The tab 56 can have any design, shape, and/or dimension deemed suitable to enable grasping of the tab 56 by the wearer to extend the extendable portion 30. The tab 56 can be grasped by the wearer of the absorbent article 10 and can be pulled in the direction indicated by the arrow (illustrated in FIGS. 1B and 2B) to extend the extendable portion 30 and create an extended region 28 for additional coverage.

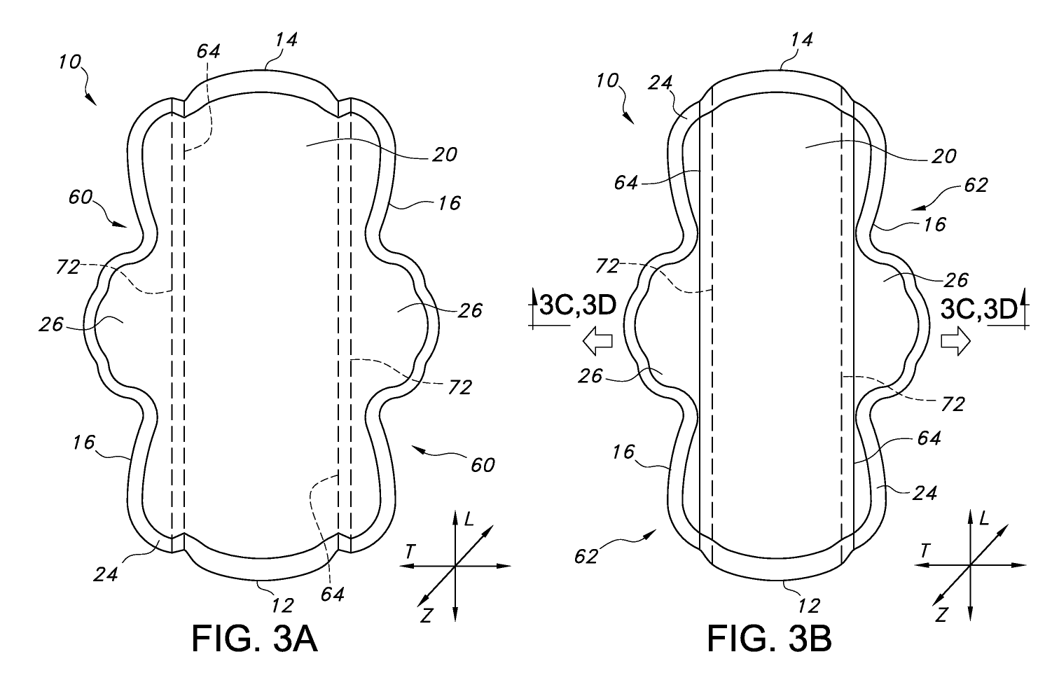

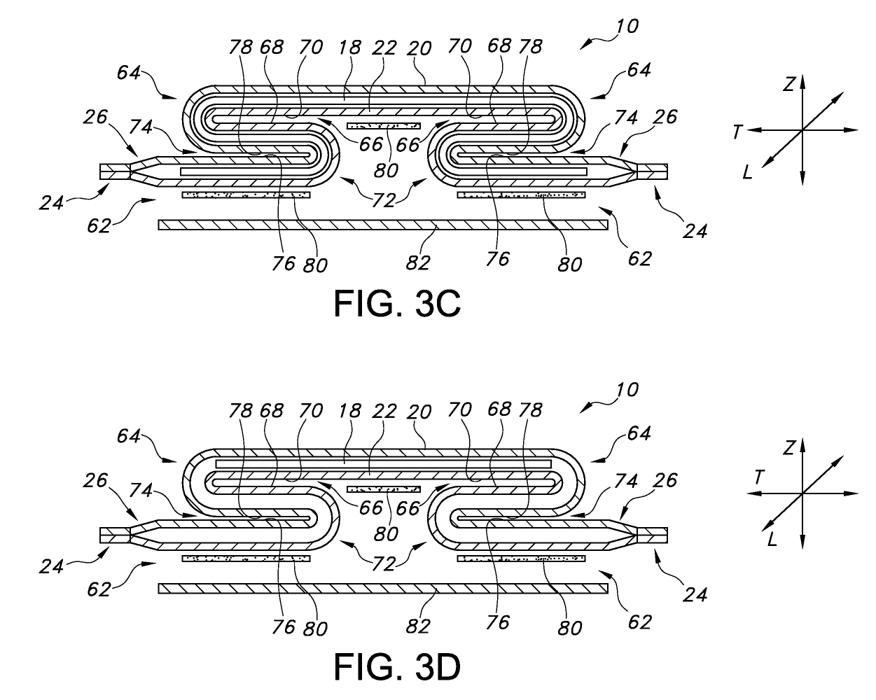

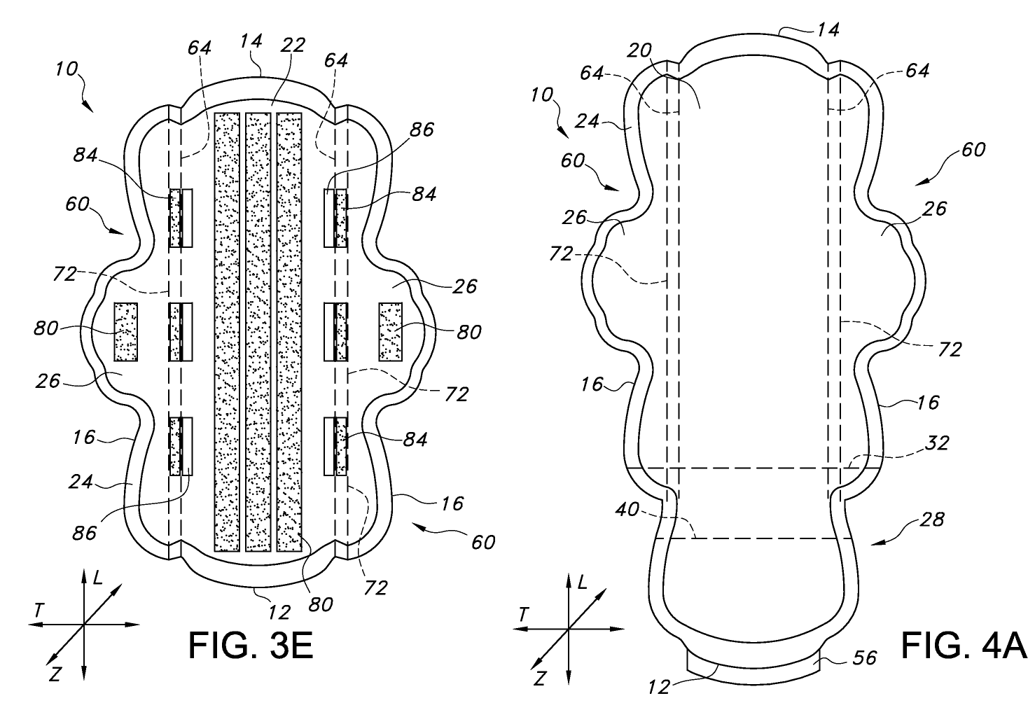

FIGS. 3A-3E provide illustrations of various views of an exemplary embodiment of an extendable absorbent article 10 having two extendable portions 62 capable of extending the absorbent article 10 in the transverse direction and forming extended regions 60. In the embodiment illustrated in FIGS. 3A-3E, the extendable portions 62 are illustrated as being constructed as an extension of at least the topsheet layer 20 and the backsheet layer 22. In FIG. 3C, the extendable portions 62 further include in their construction an extension of the absorbent core 18 while FIG. 3D provides an illustration of the absorbent article without the absorbent core 18 extending into the extendable portions 62. In the exemplary embodiment of the absorbent article 10 illustrated in FIGS. 3A-3E, the two extendable portions 62 are mirror images of each other and are each capable of extending the absorbent article 10 in a transverse direction of the absorbent article 10. As the two extendable portions 62 are mirror images of each other, they can extend the absorbent article 10 in opposing transverse directions of the absorbent article 10.

The absorbent article 10 can have a topsheet layer 20, a backsheet layer 22, an absorbent core 18, a pair of opposing transverse direction end edges, 12 and 14, a pair of opposing longitudinal direction side edges 16, and a sealed peripheral region 24 formed by bonding the peripheral edges of the topsheet layer 20 and the backsheet layer 22 together. The absorbent article 10 can have two extendable portions 62. The extendable portions 62 and, ultimately the extended regions 60, illustrated in FIGS. 3A-3E can be at each longitudinal direction side edge 16 of the absorbent article 10. Each extendable portion 62 can include at least a portion of the longitudinal direction side edges 16 of the absorbent article 10. In various embodiments in which the absorbent article 10 has a pair of wings 26, each extendable portion 62 can include at least a portion of the wings 26, respectively. Each of the extendable portions 62 can also include a portion of the sealed peripheral region 24.

Prior to usage of the absorbent article 10 by a wearer, the absorbent article 10 can be in a first configuration, such as illustrated in FIGS. 3B-3D, and can have extendable portions 62 ready for use by a wearer to extend the absorbent article 10 if so desired. The two extendable portions 62, such as illustrated in FIGS. 3B-3D, are mirror images of each other and are each independently capable of extending the absorbent article 10 in the transverse direction of the absorbent article 10, thereby increasing the first configuration first transverse width of the absorbent article 10 to a second configuration second transverse width.

As described herein, the extendable portions 62 can be incorporated into the absorbent article 10 by forming the absorbent article 10 into a fully extended configuration having the fully extended size dimensions. In the embodiment such as illustrated in FIG. 3C, the topsheet layer 20, the backsheet layer 22, and the absorbent core 18 can all be sized to the fully extended size dimension of a fully extended absorbent article 10. It is to be understood that the topsheet layer 20 and the backsheet layer 22 can still extend beyond the outermost periphery of the absorbent core 18 in order to be bonded together to form a sealed peripheral region 24. In the embodiment such as illustrated in FIG. 3D, the topsheet layer 20 and the backsheet layer 22 can extend beyond the outermost periphery of the absorbent core 18 and can both extend and both be sized to the fully extended size dimension of a fully extended absorbent article 10. The topsheet layer 20 and the backsheet layer 22 can be bonded together at their peripheral edges to form a sealed peripheral region 24. In each embodiment, a portion of the fully extended absorbent article 10 can then be folded back and under the remainder of the absorbent article 10.

The portion of the fully extended absorbent article 10 which can be folded can be the portions of the fully extended absorbent article 10 forming the extended regions 60. Incorporating a first fold into an extended region 60 can bring a portion of the extended region 60 back and under a remaining portion of the absorbent article 10. FIG. 3A provides an exemplary illustration of a potential location where a first fold, such as fold 64, can be incorporated into the extended regions 60 of the absorbent article 10. The absorbent article 10 of FIGS. 3A-3E can be capable of extending the absorbent article 10 in the transverse direction of the absorbent article 10 and, therefore, the fold, such as fold 64, can extend from the first transverse direction end edge 12 to the second transverse direction end edge 14. As a result of incorporating each first fold 64 into the absorbent article 10, an opposing pair of first regions 66 (illustrated in FIGS. 3C and 3D) can be formed wherein, for each extendable portion 62, a first portion 68 of the backsheet layer 22 can be brought into a face-to-face relationship with a second portion 70 of the backsheet layer 22.

The incorporation of each of the first folds 64 into the absorbent article 10 can re-position each of the longitudinal direction side edges to be closer in proximity to the opposite longitudinal direction side edge. Additionally, each of the first folds 64 can, in essence, become new longitudinal direction side edges for the absorbent article 10. A wearer of the absorbent article 10, however, need not extend an extendable portion 62 if they do not have a need for such an extension at the time of usage of the absorbent article 10. In such embodiments when a wearer does not extend the extendable portion 62 and wherein a first fold 64 is the longitudinal direction side edge of the absorbent article 10, the absorbent article 10 could experience leakage of body exudates as the absorbent article 10 would lack a sealed peripheral region 24 in the area of the first fold 64. To provide the absorbent article 10 with an extendable portion 62 and a sealed peripheral region 24 at the perimeter of the absorbent article 10 in a first configuration, a second fold 72 can be provided in order to re-direct a portion of the extendable portion 62 such that a portion of each of the extendable portions 62 extends beyond each of the first folds 64, respectively. As the extendable portions 62 can be constructed as an extension of at least the topsheet layer 20 and the backsheet layer 22, and as the extendable portions 62 contain a portion of the sealed peripheral region 24 of the absorbent article 10, a portion of the sealed peripheral region 24 can also now be positioned beyond each of the first folds 64 with the incorporation of the second folds 72. The absorbent article 10 can have, therefore, a sealed peripheral region 24 along the complete perimeter of the absorbent article 10 in a first configuration (as is visible in FIG. 3B). As a result of incorporating the second folds 72 in the absorbent article 10, an opposing pair of second regions 74 can be formed wherein, for each extendable portion 62, a first portion 76 of the topsheet layer 20 can be brought into a face-to-face relationship with a second portion 78 of the topsheet layer 20 (such as seen in FIGS. 3C and 3D). The two folds, 64 and 72, can create a Z-shaped or S-shaped fold pattern in each of the extendable portions 62.

As illustrated in FIGS. 3C-3E, the backsheet layer 22 of the absorbent article 10 can be provided with a garment attachment mechanism 80. The garment attachment mechanism 80 can be any operative, conventional attachment mechanism such as described with regard to the absorbent article 10 of FIGS. 1A-1D. While a pattern of lines of garment attachment mechanism 80 is illustrated in FIG. 3E, the garment attachment mechanism 80 can be provided on the backsheet layer 22 in any location and in any amount as deemed suitable to enable attachment between the absorbent article 10 and the wearer's undergarment. In various embodiments, the absorbent article 10 can utilize a peel strip layer 82 over a garment attachment mechanism 80 to protect the garment attachment mechanism 80 prior to usage of the absorbent article 10.

In various embodiments, while the backsheet layer 22 of the absorbent article 10 can be provided with a garment attachment mechanism 80, each of the first portions 68 of the backsheet layer 22 and each of the second portions 70 of the backsheet layer 22 which form each of the first regions 66 in the first configuration of the absorbent article 10 can each remain free of a garment attachment mechanism. In various embodiments, in each first region 66, a portion of at least one of the first portion 68 of the backsheet layer 22 or the second portion 78 of the backsheet layer 22 can be provided with a first region garment attachment mechanism 84. To provide for engagement between each of the first portions 68 of the backsheet layer 22 and the respective second portions 70 of the backsheet layer 22, the first portions 68 of the backsheet layer 22 can be situated over and engaged with their respective second portions 70 of the backsheet layer 22 via the first region garment attachment mechanism 84 located within each of the first regions 66. Engagement between the first portions 68 of the backsheet layer 22 and their respective second portions 70 of the backsheet layer 22 can provide for an opposing pair of extendable portions 62 which can remain in a first configuration. When a wearer of the absorbent article 10 desires to extend the extendable portions 62, at least one of the first portions 68 of the backsheet layer 22 can be dis-engaged from its respective second portion 70 of the backsheet layer 22 in a conversion of the absorbent article 10 from a first configuration to a second configuration. A first region garment attachment mechanism 84 can also help secure the extended region 60 of the absorbent article 10 to a wearer's undergarment in the event that the wearer has extended the extendable portion 62 of the absorbent article 10.

In various embodiments, to provide for engagement between each of the first portions 68 of the backsheet layer 22 and their respective second portions 70 of the backsheet layer 22 in each of the first regions 66, a first region garment attachment mechanism 84 can be positioned on a portion of each of the first portions 68 of the backsheet layer 22 and/or a portion of each of the second portions 70 of the backsheet layer 22 in each of the first regions 66. The first portions 68 of the backsheet layer 22 can then be brought into contact with their respective second portions 70 of the backsheet layer 22 and the first region garment attachment mechanisms 84 can maintain engagement between the first portions 68 and the second portions 70 of the backsheet layer 22. In various embodiments in which the first portions 68 of the backsheet layer 22 are engaged with a second portions 70 of the backsheet layer 22 via a first region garment attachment mechanism 84, it can be beneficial to dis-engage a first portion 68 of the backsheet layer 22 from a second portion 70 of the backsheet layer 22 in order to allow for extension of an individual extendable portion 62 as desired by the wearer of the absorbent article 10. To provide for such releasable engagement between one of the first portions 68 of the backsheet layer 22 and its respective second portion 70 of the backsheet layer, a portion of the first portion 68 of the backsheet layer 22 and/or a portion of the second portion 38 of the backsheet layer 22 which would be positioned opposite the first region garment attachment mechanism 84 can be provided with a corresponding first region release component 86.

For example, in various embodiments, at least a portion of a first portion 68 of the backsheet layer 22 can be provided with a first region garment attachment mechanism 84 in the form of a garment attachment adhesive and at least a portion of a second portion 70 of the backsheet layer 22 can be coated with a silicone treatment, as the first region release component 86, in a location of the second portion 70 of the backsheet layer 22 which will be opposite the first region garment attachment mechanism 84 positioned on the first portion 68 of the backsheet layer 22. When the first portion 68 of the backsheet layer 22 and its respective second portion 70 of the backsheet layer 22 are in a first configuration such as illustrated in FIGS. 3B-3D, the first region release component 86, such as the silicon treatment, is in a face-to-face relationship with the first region garment attachment mechanism 84, such as the garment attachment adhesive, positioned on the first portion 68 of the backsheet layer 22. In such embodiments, the silicone treatment which is in the face-to-face relationship with the garment attachment adhesive can prevent a permanent engagement between the first portion 68 of the backsheet layer 22 and the second portion 70 of the backsheet layer 22. The first portion 68 of the backsheet layer 22 can, therefore, be capable of dis-engaging from the second portion 70 of the backsheet layer 22 when the wearer desires to extend the extendable portion 62.

Referring to FIG. 3E, a bottom view of an exemplary absorbent article 10 extended in a fully extended configuration is illustrated. FIG. 3E provides an illustration of potential locations where folds, such as folds 64 and 72, can be incorporated into the absorbent article 10. In various embodiments, the fully extended configuration of an absorbent article 10 can exist such as, for example, during the manufacturing process prior to folding the absorbent article 10 into a first configuration and prior to incorporating folds, such as folds 64 and 72, into the absorbent article 10. In various embodiments, the fully extended configuration can exist, such as, for example, during usage when a wearer has altered the configuration of the absorbent article 10 from the first configuration to a second configuration by extending the extendable portion 62 into an extended region 60.

As illustrated in FIG. 3E, a portion of the backsheet layer 22 which will form the first portions 68 of the backsheet layer 22 in the first region 66 when the absorbent article 10 is in a first configuration can be provided with a first region garment attachment mechanism 84 and a portion of the backsheet layer 22 which will form the second portions 70 of the backsheet layer 22 in the first region 66 when the absorbent article 10 is in a first configuration can be provided with a corresponding first region release component 86. When the absorbent article 10 is in a first configuration, the first region garment attachment mechanism 84 and the first region release component 86 can align with each other and can be in a face-to-face relationship. Such alignment can prevent a permanent attachment between the first portions 68 and the second portions 70 of the backsheet layer 22. While the first portions 68 have been illustrated with a first region garment attachment mechanism 84 and the second portions 70 have been illustrated with a first region release component 86, it is to be understood that each of the first region garment attachment mechanism 84 and the first region release component 86 can be located on either or both of the first portions 68 and/or second portions 70 of the backsheet layer 22 in the first regions 66.

In various embodiments, each of the first portions 68 and the second portions 70 of the backsheet layer 22 can be provided with a garment attachment mechanism. In such embodiments in which both the first portions 68 and the second portions 70 of the backsheet layer 22 have a garment attachment mechanism, each of the first portions 68 and second portions 70 of the backsheet layer 22 can have a corresponding release component, positioned opposite a garment attachment mechanism on the opposing portion of the backsheet layer 22, which can allow for the garment attachment mechanisms to dis-engage and allow for the unfolding of the extendable portions 62. Therefore, a release component, positioned on the second portions 70 of the backsheet layer 22 can allow for a garment attachment mechanism located on the first portions 68 of the backsheet layer 22 to dis-engage. Similarly, a release component positioned on the first portions 68 of the backsheet layer 22 can allow for a garment attachment mechanism located on the second portions 70 of the backsheet layer 22 to dis-engage. For example, in various embodiments, the first portions 68 of the backsheet layer 22 can be provided with a garment attachment mechanism in the form of a garment attachment adhesive and the second portions 70 of the backsheet layer 22 can be provided with a garment attachment mechanism in the form of a garment attachment adhesive. In such embodiments, each of the first portions 68 of the backsheet layer 22 and the second portions 70 of the backsheet layer 22 can also be at least partially coated with a silicone treatment as the corresponding release component. In such embodiments, the silicone treatment on the first portions 68 of the backsheet layer 22 can align in a face-to-face relationship with a garment attachment adhesive on the second portions 70 of the backsheet layer 22 and a silicone treatment on the second portions 70 of the backsheet layer 22 can align in a face-to-face relationship with a garment attachment adhesive on the first portions 68 of the backsheet layer 22. Such alignment can prevent a permanent attachment between the two portions, first portions 68 and second portions 70, of the backsheet layer 22.

In various embodiments, the first region garment attachment mechanism 84 and the corresponding first region release component 86 can be provided in a pattern arrangement. In various embodiments, the first region garment attachment mechanism 84 can be provided in any pattern as deemed suitable. Exemplary patterns can include, but are not limited to, lines, swirls, dots, circles, ovals, and diamonds. In such embodiments, the corresponding first region release component 86 can be provided in a pattern arrangement which can mirror the pattern of the first region garment attachment mechanism 84.

The absorbent article 10 can be provided with wings 26. The wings 26 can provide a location for a wearer of the absorbent article 10 to grab the extendable portions 62 and pull the extendable portions 62 in the direction indicated by the arrows (in FIG. 3B) in order to create extended regions 60 of the absorbent article 10. In the event that the absorbent article 10 is not provided with wings 26, to assist the wearer with extending the extendable portions 62, the extendable portions can be provided with a tab. The tab can be of an integral unitary construction with the extendable portions 62 or the tab can be a separate element bonded to the absorbent article 10. The tab can be formed from materials similar to the topsheet layer 20, the backsheet layer 22 or combinations of these materials. The tab can have any design, shape, and/or dimension deemed suitable to enable grasping of the tab by the wearer to extend the extendable portions 62. The tab can be grasped by the wearer of the absorbent article 10 and can be pulled in the direction indicated by the arrow (in FIG. 3B) to extend the extendable portions 62 and create an extended region 60 for additional coverage.

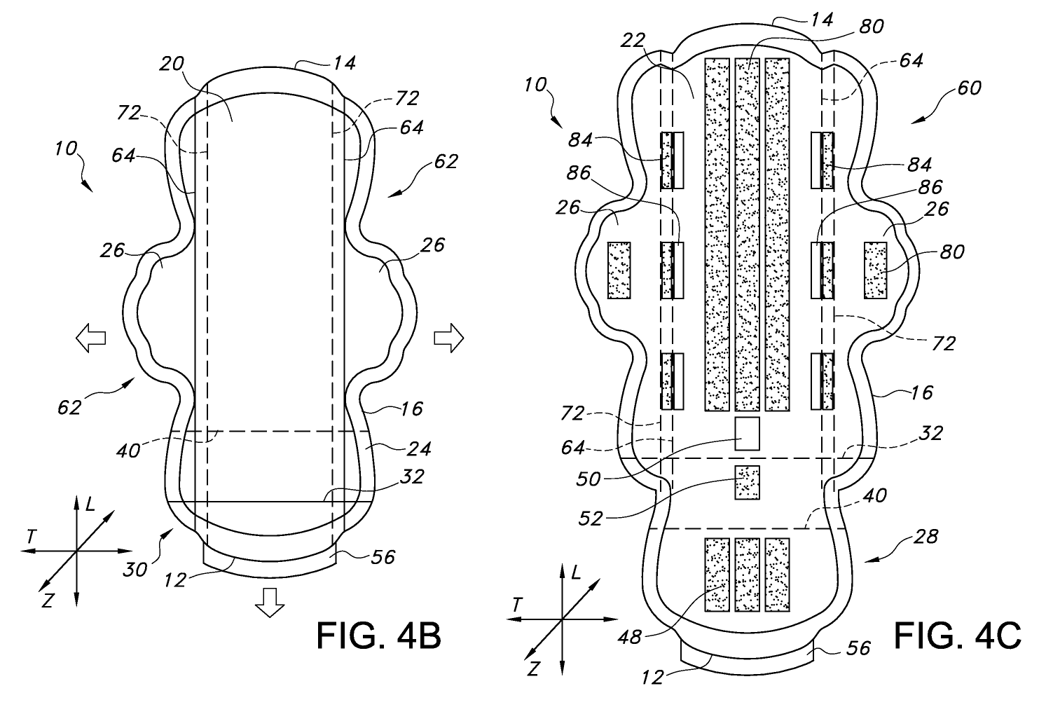

FIGS. 4A-4C provide illustrations of various views of an exemplary embodiment of an extendable absorbent article 10 having one extendable portion 30, which is capable of extending the absorbent article 10 in the longitudinal direction of the absorbent article 10, and two extendable portions 62, which are capable of extending the absorbent article 10 in the transverse direction of the absorbent article 10. FIG. 4A provides a top view illustration of an exemplary embodiment of an extendable absorbent article 10 in a fully extended configuration wherein the absorbent article 10 has an extended region 28 in the longitudinal direction of the absorbent article 10 and a pair of extended regions 60 in the transverse direction of the absorbent article 10. As can be seen from FIGS. 4A-4C, the absorbent article 10 of FIGS. 4A-4C can be a combination of the absorbent article 10 of FIGS. 1A-1D and the absorbent article 10 of FIGS. 3A-3E.

In various embodiments, the longitudinal direction extendable portion 30 can be constructed as an extension of the topsheet layer 20 and the backsheet layer 22 of the absorbent article 10. In various embodiments, the longitudinal direction extendable portion 30 can be constructed as an extension of the topsheet layer 20, the backsheet layer 22, and an absorbent material. In various embodiments, the transverse direction extendable portions 62 can be constructed as extensions of the topsheet layer 20 and the backsheet layer 22 of the absorbent article 10. In various embodiments, the transverse direction extendable portions 62 can be constructed as extensions of the topsheet layer 20, the backsheet layer 22, and an absorbent material.

In various embodiments, the extendable portion 30 and the pair of extendable portions 62 can be constructed as an extension of the topsheet layer 20 and the backsheet layer 22 of the absorbent article 10. In various embodiments, the extendable portion 30 and the pair of extendable portions 62 can be constructed as an extension of the topsheet layer 20, the backsheet layer 22, and an absorbent material. In various embodiments, the extendable portion 30 can be constructed as an extension of the topsheet layer 20 and the backsheet layer 22 of the absorbent article 10 and the pair of extendable portions 62 can be constructed as an extension of the topsheet layer 20, the backsheet layer 22, and an absorbent material. In various embodiments, the extendable portion 30 can be constructed as an extension of the topsheet layer 20, the backsheet layer 22, and an absorbent material, and the pair of extendable portions 62 can be constructed as an extension of the topsheet layer 20 and the backsheet layer 22 of the absorbent article 10.

In various embodiments, in the first configuration of the absorbent article 10, the extendable portion 30 can overlay the pair of extendable portions 62. For example, in the first configuration, at least a portion of each of the extendable portions 62 can be positioned in a portion of the first region 34 of the absorbent article 10. In various embodiments, in the first configuration of the absorbent article 10, the extendable portions 62 can overlay the extendable portion 30. For example, in the first configuration, at least a portion of the extendable portion 30 can be positioned within a portion of the first region 66 of each of the extendable portions 62. In various embodiments, in the first configuration of the absorbent article 10, the absorbent article 10 can be sized such that there is no overlap between the extendable portion 30 and the pair of extendable portions 62.

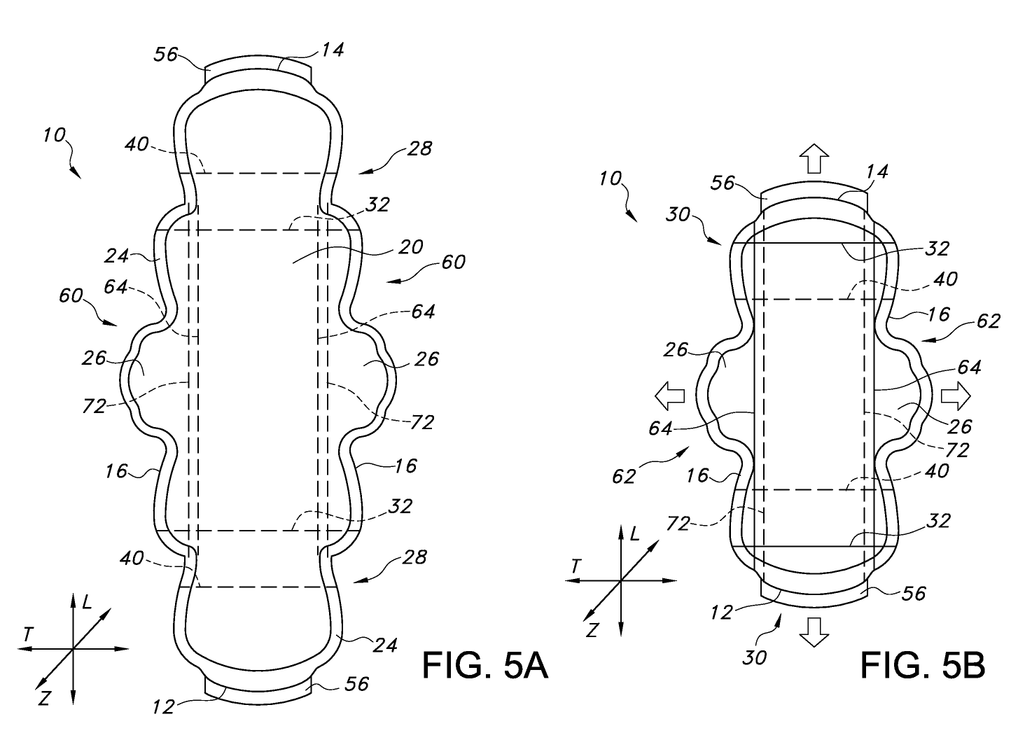

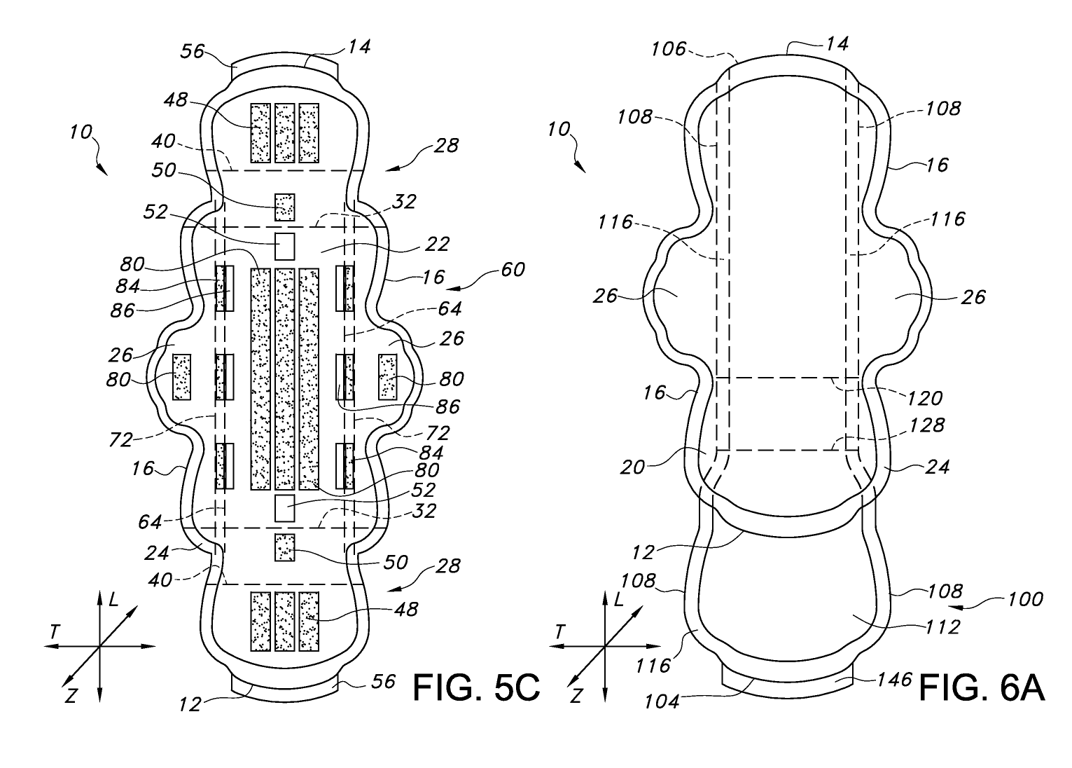

FIGS. 5A-5C provide illustrations of various views of an exemplary embodiment of an extendable absorbent article 10 having two extendable portions 30, which are capable of extending the absorbent article 10 in the longitudinal direction of the absorbent article 10, and two extendable portions 62, which are capable of extending the absorbent article 10 in the transverse direction of the absorbent article 10. FIG. 5A provides a top view illustration of an exemplary embodiment of an extendable absorbent article 10 in a fully extended configuration wherein the absorbent article 10 has two extended regions 28 in the longitudinal direction of the absorbent article 10 and a pair of extended regions 60 in the transverse direction of the absorbent article 10. As can be seen from FIGS. 5A-5C, the absorbent article 10 of FIGS. 5A-5C can be a combination of the absorbent article 10 of FIGS. 2A-2D and the absorbent article 10 of FIGS. 3A-3E.

In various embodiments, the longitudinal direction extendable portions 30 can be constructed as extensions of the topsheet layer 20 and the backsheet layer 22 of the absorbent article 10. In various embodiments, the longitudinal direction extendable portions 30 can be constructed as extensions of the topsheet layer 20, the backsheet layer 22, and an absorbent material. In various embodiments, the transverse direction extendable portions 62 can be constructed as extensions of the topsheet layer 20 and the backsheet layer 22 of the absorbent article 10. In various embodiments, the transverse direction extendable portions 62 can be constructed as extensions of the topsheet layer 20, the backsheet layer 22, and an absorbent material.

In various embodiments, the pair of extendable portions 30 and the pair of extendable portions 62 can be constructed as extensions of the topsheet layer 20 and the backsheet layer 22 of the absorbent article 10. In various embodiments, the pair of extendable portions 30 and the pair of extendable portions 62 can be constructed as extensions of the topsheet layer 20, the backsheet layer 22, and an absorbent material. In various embodiments, the pair of extendable portions 30 can be constructed as extensions of the topsheet layer 20 and the backsheet layer 22 of the absorbent article 10 and the pair of extendable portions 62 can be constructed as an extension of the topsheet layer 20, the backsheet layer 22, and an absorbent material. In various embodiments, the pair of extendable portions 30 can be constructed as extensions of the topsheet layer 20, the backsheet layer 22, and an absorbent material, and the pair of extendable portions 62 can be constructed as an extension of the topsheet layer 20 and the backsheet layer 22 of the absorbent article 10.

In various embodiments, in the first configuration of the absorbent article 10, the pair of extendable portions 30 can overlay the pair of extendable portions 62. For example, in the first configuration, at least a portion of each of the extendable portions 62 can be positioned in a portion of each of the first regions 34 of the absorbent article 10. In various embodiments, in the first configuration of the absorbent article 10, the extendable portions 62 can overlay the extendable portions 30. For example, in the first configuration, at least a portion of the extendable portions 30 can be positioned within a portion of the first region 66 of each of the extendable portions 62. In various embodiments, in the first configuration of the absorbent article 10, the absorbent article 10 can be sized such that there is no overlap between the extendable portions 30 and the pair of extendable portions 62.

As described herein, in various embodiments, an extendable portion can be constructed of materials similar to the topsheet layer 20, the backsheet layer 22, and in various embodiments an absorbent material such as any absorbent material described herein as suitable for the absorbent core 18 or fluid intake layer. In such embodiments, at least a portion of the extendable portion can be permanently bonded with a layer of the absorbent article 10, such as, for example, the backsheet layer 22 of the absorbent article 10.

An extendable portion constructed of materials similar to the topsheet layer 20, the backsheet layer 22, and in various embodiments an absorbent material, and which is then permanently bonded with a layer of the absorbent article 10, such as, for example, the backsheet layer 22 of the absorbent article 10, can be constructed as a laminate and can have a liquid permeable layer, a liquid impermeable layer, and in various embodiments an absorbent material can be contained between the liquid permeable layer and the liquid impermeable layer.

The liquid permeable layer can be formed from any material described herein as suitable for the topsheet layer 20. The liquid permeable layer can be a single layer of material, or alternatively, can be multiple layers that have been laminated together. The liquid permeable layer can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a liquid permeable layer.

In various embodiments, the liquid permeable layer can be constructed from various nonwoven webs such as meltblown webs, spunbond webs, hydroentangled spunlace webs, or through air bonded carded webs. Examples of suitable liquid permeable layer materials can include, but are not limited to, natural fiber webs (such as cotton), rayon, bonded carded webs of polyester, polypropylene, polyethylene, nylon, or other heat-bondable fibers, polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable liquid permeable layer can be a bonded carded web made of polypropylene and polyethylene such as that obtainable from Sandler Corporation, Germany.

In various embodiments, the liquid permeable layer can have a basis weight ranging from about 5, 10, 15, 20 or 25 gsm to about 50, 100, 120, 125 or 150 gsm. For example, in an embodiment, a liquid permeable layer can be constructed from a through air bonded carded web having a basis weight ranging from about 15 gsm to about 100 gsm. In another example, a liquid permeable layer can be constructed from a through air bonded carded web having a basis weight from about 20 gsm to about 50 gsm, such as a through air bonded carded web that is readily available from nonwoven material manufacturers, such as Xiamen Yanjan Industry, Beijing DaYuan Nonwoven Fabrics and others.

In various embodiments, the liquid permeable layer can be at least partially hydrophilic. In various embodiments, a portion of the liquid permeable layer can be hydrophilic and a portion of the liquid permeable layer can be hydrophobic. In various embodiments, the portions of the liquid permeable layer which can be hydrophobic can be either an inherently hydrophobic material or can be a material treated with a hydrophobic coating.

The liquid impermeable layer can be formed from any material described herein as suitable for the backsheet layer 22. The liquid impermeable layer can permit the passage of air or vapor out of the absorbent article 10 while still blocking the passage of liquids. Any liquid impermeable material may generally be utilized to form the liquid impermeable layer. The liquid impermeable layer can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the liquid impermeable layer can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a liquid impermeable layer can be a polyethylene film such as that obtainable from Pliant Corporation, Schaumburg, Ill., USA. Another example can include calcium carbonate-filled polypropylene film. In still another embodiment, the liquid impermeable layer can be a hydrophobic nonwoven material with water barrier properties such as a nonwoven laminate, an example of which can be a spunbond, meltblown, meltblown, spunbond, four-layered laminate. The liquid impermeable layer can, therefore, be of a single or multiple layer construction, such as of multiple film layers or laminates of film and nonwoven fibrous layers.

In various embodiments, the extendable portion can contain an absorbent material positioned between the liquid permeable layer and the liquid impermeable layer. In such embodiments, the absorbent material of the protection layer can be formed from any absorbent material such as, for example, materials described herein as suitable for the absorbent core 18 or fluid intake layer.

In various embodiments in which an extendable portion can be constructed as a laminate of a liquid permeable layer, a liquid impermeable layer, and in various embodiments an absorbent material such as any material described herein as suitable for the absorbent core 18 or fluid intake layer, the manufacture of the absorbent article 10 can include a step of forming the laminate which will ultimately become the extendable portion and a step of permanently attaching the laminate to a layer of an absorbent article 10, such as, for example, the backsheet layer 22 of the absorbent article 10. To form the laminate, the materials utilized for the liquid permeable layer and the liquid impermeable layer (and absorbent material if desired) of the laminate can be sized to have the fully extended size dimensions for a fully extended absorbent article 10. To form the laminate the liquid permeable layer and the liquid impermeable layer can be bonded together at their periphery, either entirely or partially, using known bonding techniques to form a sealed peripheral region. The laminate can have, therefore, a liquid permeable layer, a liquid impermeable layer, an opposing pair of transverse direction end edges and an opposing pair of longitudinal direction side edges. If desired, the laminate can also have an absorbent material positioned between the liquid permeable layer and the liquid impermeable layer. At least a portion of the laminate can be permanently attached to a layer of the absorbent article 10, such as, for example, the backsheet layer 22 of the absorbent article 10. A portion of the laminate can remain free from permanent attachment and available to be further formed into an extendable portion of the absorbent article 10. In various embodiments, when the laminate is permanently attached to a layer of the absorbent article 10, the laminate can be in an unfolded configuration and at least a portion of the laminate can extend in a fully extended configuration beyond at least one of the transverse direction end edges and/or longitudinal direction side edges, of the absorbent article 10. The portion of the laminate extending beyond at least one of the edges of the absorbent article 10 can be an extended region of the absorbent article. A portion of the fully extended laminate can then be folded back and under the remainder of the absorbent article 10.

The portion of the fully extended laminate which can be folded can be the portion of the fully extended laminate forming the extended region. Incorporating a first fold into an extended region can bring a portion of the extended region back and under a remaining portion of the absorbent article 10. The folding of the portion of the laminate can initiate the placement of the laminate and the absorbent article into a first configuration. The folded portion of the laminate can form an extendable portion which can later be utilized by a wearer of the absorbent article as desired based upon the wearer's needs. The wearer can manipulate an extendable portion (e.g. unfolding the extendable portion) to extend the dimensions of the absorbent article as desired to meet their needs. If an extendable portion is not needed by the wearer during use, the extendable portion can remain in the folded, first configuration.

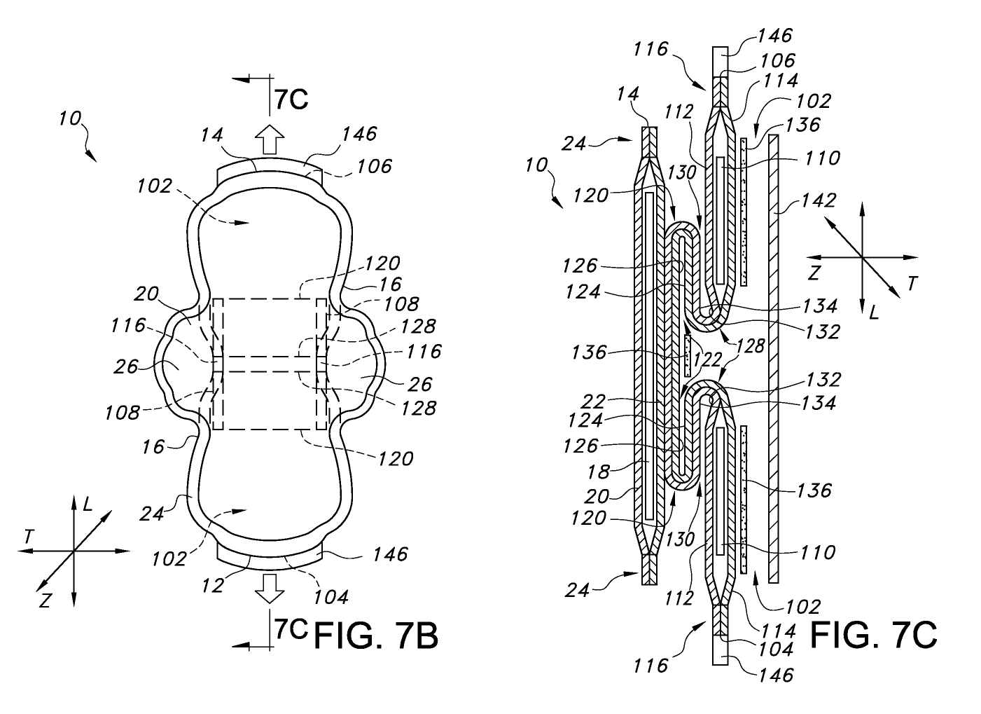

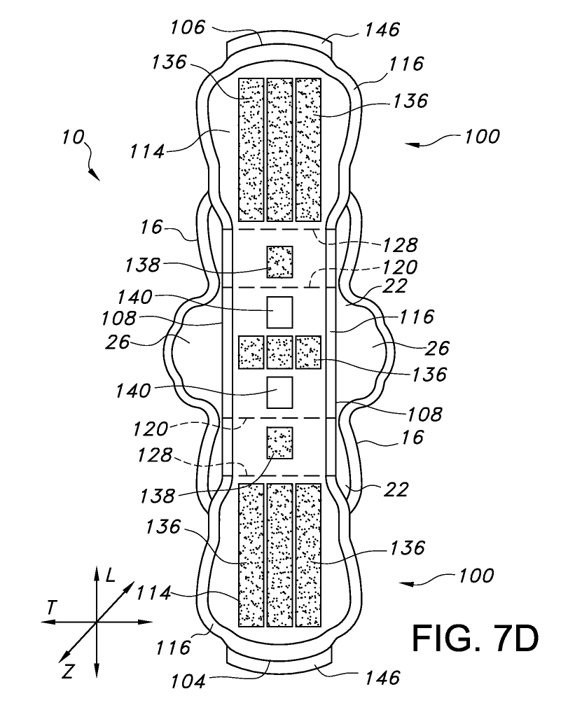

FIGS. 6A-6D and FIGS. 7A-7D provide illustrations of various views of exemplary embodiments of extendable absorbent articles 10 having an extendable portion 102 which can extend the absorbent article 10 in the longitudinal direction of the absorbent article 10. FIGS. 6A-6D provide illustrations of various views of an exemplary absorbent article 10 having one extendable portion 102 capable of extending the absorbent article 10 in the longitudinal direction and forming extended regions 100. In the embodiment illustrated in FIGS. 6A-6D, the extendable portion 102 is illustrated as being constructed as a laminate of a liquid permeable layer 112, a liquid impermeable layer 114 and an absorbent material 110. FIGS. 7A-7D provide illustrations of various views of an exemplary absorbent article 10 having two extendable portions 102. The two extendable portions 102 are mirror images of each and are capable of extending the absorbent article 10 in the longitudinal direction and forming extended region 100. The absorbent article 10 illustrated in FIGS. 7A-7D is similar to the absorbent article illustrated in FIGS. 6A-6D in that each extendable portion of the absorbent article 10 illustrated in FIGS. 7A-7D is illustrated as being constructed as a laminate of a liquid permeable layer 112, a liquid impermeable layer 114 and an absorbent material 110. As described herein, an extendable portion 102 need not include in its construction any absorbent material. Thus, while the absorbent article 10 of FIGS. 6A-6D and the absorbent article 10 of FIGS. 7A-7D are illustrated as having at least one extendable portion 102 including an absorbent material 110 within the construction of the extendable portion 102, an absorbent article 10 can be manufactured similar to those of FIGS. 6A-6D and 7A-7D without an absorbent material within the construction of the extendable portion 102.

Referring to FIGS. 6A-6D and 7D-7D, the absorbent article 10 can have a topsheet layer 20, a backsheet layer 22, an absorbent core 18, a pair of opposing transverse direction end edges, 12 and 14, a pair of opposing longitudinal direction side edges 16, and a sealed peripheral region 24 formed by bonding the peripheral edges of the topsheet layer 20 and the backsheet layer 22 together. The absorbent article 10 illustrated in FIGS. 6A-6D can have one extendable portion 102 constructed as a laminate of a liquid permeable layer 112, a liquid impermeable layer 114 and an absorbent material 110. The laminate can further have a pair of opposing transverse direction end edges, 104 and 106, a pair of opposing longitudinal direction side edges 108, and a sealed peripheral region 116. A portion of the laminate can be permanently attached to a layer of the absorbent article 10, such as, for example the backsheet layer 22 of the absorbent article 10. In the embodiment illustrated in FIGS. 6A-6D, at least a portion of the laminate can be permanently attached to a layer of the absorbent article 10, such as for example, the backsheet layer 22, such that one of the transverse direction end edges of the laminate can be substantially aligned with one of the transverse direction end edges of the absorbent article. In the embodiment illustrated in FIGS. 6A-6D, transverse direction end edge 106 of the laminate can be aligned with transverse direction end edge 14 of the absorbent article 10. In such an exemplary embodiment, a portion of the laminate can remain free from permanent attachment in order to form the extendable portion 102. Referring to FIGS. 7A-7D, the absorbent article 10 illustrated can have two extendable portions 102 constructed from a common laminate of a liquid permeable layer 112, a liquid impermeable layer 114 and an absorbent material 110. The laminate can further have a pair of opposing transverse direction end edges, 104 and 106, a pair of opposing longitudinal direction side edges 108, and a sealed peripheral region 116. A portion of the laminate can be permanently attached to a layer of the absorbent article 10, such as, for example the backsheet layer 22 of the absorbent article 10. In the embodiment illustrated in FIGS. 7A-7D, at least a portion of the laminate can be permanently attached to a layer of the absorbent article 10, such as for example, the backsheet layer 22, such that an area of the laminate at the longitudinal midpoint of the laminate can be permanently attached to the backsheet layer 22 of the absorbent article. In such an exemplary embodiment, the remaining portions of the laminate can remain free from permanent attachment in order to form the extendable portions 102.

Prior to usage of the absorbent article 10 by a wearer, the absorbent article 10 can be in a first configuration, such as illustrated in FIGS. 6B, 6C, 7B and 7C, and can have an extendable portion 102 (FIGS. 6B and 6C) or an opposing pair of extendable portions 102 (FIGS. 7B and 7C) ready for use by a wearer to extend the absorbent article 10 if so desired. The extendable portion 102 can be capable of extending the absorbent article 10 in the longitudinal direction of the absorbent article 10, thereby increasing the first configuration first longitudinal length of the absorbent article 10 to a second configuration second longitudinal length. The two extendable portions 102 illustrated in FIGS. 7B and 7C are mirror images of each other and are each independently capable of extending the absorbent article 10 in the longitudinal direction of the absorbent article 10, thereby, increasing the first configuration first longitudinal length of the absorbent article 10 to a second configuration second longitudinal length.

As described herein, each extendable portion 102 of the absorbent article 10 illustrated in FIGS. 6A-6D and 7A-7D can be incorporated into the absorbent article 10 by forming the laminate which will ultimately become the extendable portion 102 and a step of permanently attaching the laminate to a layer of an absorbent article 10, such as, for example, the backsheet layer 22 of the absorbent article 10. To form the laminate, the materials utilized for the liquid permeable layer 112 and the liquid impermeable layer 114 (and absorbent material 110 if desired) of the laminate can be sized to have the fully extended size dimensions desired for a fully extended absorbent article 10. To form the laminate the liquid permeable layer 112 and the liquid impermeable layer 114 can be bonded together at their periphery, either entirely or partially, using known bonding techniques to form a sealed peripheral region. The laminate can have, therefore, a liquid permeable layer 112, a liquid impermeable layer 114, an opposing pair of transverse direction end edges, 104 and 106, and an opposing pair of longitudinal direction side edges 108. If desired, the laminate can also have an absorbent material 110 positioned between the liquid permeable layer 112 and the liquid impermeable layer 114. At least a portion of the laminate can be permanently attached to a layer of the absorbent article 10, such as, for example, the backsheet layer 22 of the absorbent article 10 and a portion of the laminate can remain free from permanent attachment and available to be further formed into an extendable portion 102 of the absorbent article 10. In various embodiments, when the laminate is permanently attached to a layer of the absorbent article 10, the laminate can be in an unfolded configuration and at least a portion of the laminate can extend in the fully extended configuration beyond at least one of the transverse direction end edges, 12 and/or 14, and/or longitudinal direction side edges 16, of the absorbent article 10. The portion of the laminate extending beyond at least one of the edges, 12, 14 and/or 16, of the absorbent article 10 can be an extended region 100 of the absorbent article 10. A portion of the fully extended laminate can then be folded back and under the remainder of the absorbent article 10.

The portion of the fully extended laminate which can be folded can be the portion of the fully extended laminate forming the extended region 100. Incorporating a first fold into an extended region 100 can bring a portion of the extended region 100 back and under a remaining portion of the absorbent article 10. The folding of the portion of the laminate can initiate the placement of the laminate and the absorbent article 10 into a first configuration. The folded portion of the laminate can form an extendable portion 102 which can later be utilized by a wearer of the absorbent article as desired based upon the wearer's needs. The wearer can manipulate an extendable portion 102 (e.g. unfolding the extendable portion 102) to extend the dimensions of the absorbent article 10 as desired to meet their needs. If an extendable portion 102 is not needed by the wearer during use, the extendable portion 102 can remain in the folded, first configuration.

FIGS. 6A and 7A provide exemplary illustrations of a potential location where a first fold, such as fold 120, can be incorporated into the extended region 100 of the laminate. The absorbent article 10 of FIGS. 6A-6D and 7A-7D can be capable of extending in the longitudinal direction of the absorbent article 10 and, therefore, the fold, such as fold 120, can extend from a first of the longitudinal direction side edges 108 of the laminate to the second of the longitudinal direction side edges 108 of the laminate. As a result of incorporating first fold 120 into the laminate, a first region 122 (illustrated in FIGS. 6C and 7C) can be formed wherein a first portion 124 of the liquid impermeable layer 114 can be brought into a face-to-face relationship with a second portion 126 of the liquid impermeable layer 114.

The incorporation of the first fold 120 into the laminate can re-position one of the transverse direction end edges, 104 or 106, to be closer in proximity to the opposite transverse direction end edge, 106 or 104, respectively, of the laminate. To provide the absorbent article 10 with an extendable portion 102 which can be easier for a wearer of the absorbent article 10 to manipulate and unfold, a second fold 128 can be provided in order to re-direct a portion of the extendable portion 102. As a result of incorporating the second fold 128 in the laminate, a second region 130 can be formed wherein a first portion 132 of the liquid permeable layer 112 can be brought into a face-to-face relationship with a second portion 134 of the liquid permeable layer 112 (such as seen in FIGS. 6C and 7C). A portion of the liquid permeable layer 112 can also be brought into a face-to-face relationship with the backsheet layer 22 of the absorbent article 10. The two folds, 120 and 128, can create a Z-shaped or S-shaped fold pattern in the extendable portion 102.

As illustrated in FIGS. 6C, 6D, 7C and 7D, the liquid impermeable layer 114 of the laminate of the absorbent article 10 can be provided with a garment attachment mechanism 136. The garment attachment mechanism 136 can be any operative, conventional attachment mechanism, examples of which can include, but are not limited to, adhesives, cohesives, inter-engaging mechanical fastener systems or the like, as well as combinations thereof. For example, in various embodiments, the garment attachment mechanism 136 can include a garment attachment adhesive. Examples of suitable garment attachment adhesives and patterns include, but are not limited to, those described in DE700225U1, U.S. Pat. No. 3,881,490 to Whitehead, et al., U.S. Pat. No. 3,913,580 to Ginocchio, U.S. Pat. No. 4,337,772 to Roeder, et al., GB1349962, and U.S. Publication No. 2007/0073255 to Thomas, et al., each of which are hereby incorporated by reference thereto in its entirety. While a pattern of lines of a garment attachment mechanism 136 is illustrated in FIGS. 6D and 7D, the garment attachment mechanism 136 can be provided on the liquid impermeable layer 114 in any location and in any amount as deemed suitable to enable attachment between the absorbent article 10 and the wearer's undergarment. In various embodiments, the absorbent article 10 can utilize a peel strip layer 142 over a garment attachment mechanism 136 to protect the garment attachment mechanism 136 prior to usage of the absorbent article 10.

Referring to FIGS. 6C, 6D, 7C, and 7D, in various embodiments, while the liquid impermeable layer 114 of the laminate can be provided with a garment attachment mechanism 136, the first portion 124 of the liquid impermeable layer 114 and the second portion 126 of the liquid impermeable layer 114 which form the first region 122 in the first configuration of the absorbent article 10 can remain free of a garment attachment mechanism. In various embodiments, a portion of at least one of the first portion 124 of the liquid impermeable layer 114 or the second portion 126 of the liquid impermeable layer can be provided with a first region garment attachment mechanism 138. To provide for engagement between the first portion 124 of the liquid impermeable layer 114 and the second portion 126 of the liquid impermeable layer 114 in the first region 122, the first portion 124 of the liquid impermeable layer 114 can be situated over and engaged with the second portion 126 of the liquid impermeable layer 114 via the first region garment attachment mechanism 138 located within the first region 122. Engagement between the first portion 124 of the liquid impermeable layer 114 and the second portion 126 of the liquid impermeable layer 114 can provide for an extendable portion 102 which can remain in a first configuration. When a wearer of the absorbent article 10 desires to extend the extendable portion 102, the first portion 124 of the liquid impermeable layer 114 can be dis-engaged from the second portion 126 of the liquid impermeable layer in a conversion of the absorbent article 10 from a first configuration to a second configuration. A first region garment attachment mechanism 138 can also help secure the extended region 100 of the absorbent article 10 to a wearer's undergarment in the event that the wearer has extended the extendable portion 102 of the absorbent article 10.

In various embodiments in which the first portion 124 of the liquid impermeable layer 114 is engaged with a second portion 126 of the liquid impermeable layer 114 via a first region garment attachment mechanism 138, it can be beneficial to dis-engage the first portion 124 of the liquid impermeable layer 114 from the second portion 126 of the liquid impermeable layer 114 in order to allow for extension of the extendable portion 102 as desired by the wearer of the absorbent article 10. To provide for such releasable engagement between the first portion 124 of the liquid impermeable layer 114 and the second portion 126 of the liquid impermeable layer 114, a portion of the first portion 124 of the liquid impermeable layer 114 and/or a portion of the second portion 126 of the liquid impermeable layer 114 which would be positioned opposite the first region garment attachment mechanism 138 can be provided with a corresponding first region release component 140. For example, in various embodiments, at least a portion of the first portion 124 of the liquid impermeable layer 114 can be provided with a first region garment attachment mechanism 138 in the form of a garment attachment adhesive and at least a portion of the second portion 126 of the liquid impermeable layer 114 can be coated with a silicone treatment, as the first region release component 140, in a location of the second portion 126 of the liquid impermeable layer 114 which will be opposite the first region garment attachment mechanism 138 positioned on the first portion 124 of the liquid impermeable layer 114. When the first portion 124 of the liquid impermeable layer 114 and the second portion 126 of the liquid impermeable layer are in a first configuration such as illustrated in FIGS. 6C and 7C, the first region release component 140, such as the silicone treatment, is in a face-to-face relationship with the first region garment attachment mechanism 138, such as the garment attachment adhesive. In such embodiments, the silicone treatment which is in the face-to-face relationship with the garment attachment adhesive can prevent a permanent engagement between the first portion 124 of the liquid impermeable layer 114 and the second portion 126 of the liquid impermeable layer 114. The first portion 124 of the liquid impermeable layer 114, therefore, can be capable of dis-engaging from the second portion 126 of the liquid impermeable layer 114 when the wearer of the absorbent article 10 desires to extend the extendable portion 102.

Referring to FIGS. 6D and 7D, a bottom view of an exemplary absorbent article 10 extended in a fully extended configuration is illustrated. FIGS. 6D and 7D provide illustrations of potential locations where folds, such as folds 120 and 128, can be incorporated into the laminate. In various embodiments, the fully extended configuration of an absorbent article 10 can exist such as, for example, during the manufacturing process prior to incorporating folds, such as folds 120 and 128, into the laminate. In various embodiments, the fully extended configuration can exist, such as, for example, during usage when a wearer has altered the configuration of the absorbent article 10 from the first configuration to a second configuration by extending the extendable portion(s) 102 into extended region(s) 100.

As illustrated in FIGS. 6D and 7D, a portion of the liquid impermeable layer 114 which will form a first portion 124 of the liquid impermeable layer 114 in a first region 122 when the absorbent article 10 is in a first configuration can be provided with a first region garment attachment mechanism 138 and a portion of the liquid impermeable layer 114 which will form a second portion 126 of the liquid impermeable layer 114 in a first region 122 when the absorbent article 10 is in a first configuration can be provided with a corresponding first region release component 140. When the absorbent article 10 is in a first configuration, the first region garment attachment mechanism 138 and the first region release component 140 can align with each other and can be in a face-to-face relationship. Such alignment can prevent a permanent attachment between the first portion 124 and the second portion 126 of the liquid impermeable layer 114. While the first portion 124 has been illustrated with a first region garment attachment mechanism 138 and the second portion 126 has been illustrated with a first region release component 140, it is to be understood that each of the first region garment attachment mechanism 138 and the first region release component 140 can be located on either or both of the first portion 124 and/or second portion 126 of the liquid impermeable layer 114 in the first region 122.

In various embodiments, each of the first portion 124 and the second portion 126 of the liquid impermeable layer 114 can be provided with a garment attachment mechanism. In such embodiments in which both the first portion 124 and the second portion 126 of the liquid impermeable layer 114 have a garment attachment mechanism, each of the first portion 124 and second portion 126 of the liquid impermeable layer 114 can have a corresponding release component, positioned opposite a garment attachment mechanism on the opposing portion of the liquid impermeable layer 114, which can allow for the garment attachment mechanisms to dis-engage and allow for the unfolding of the extendable portion 102. Therefore, a release component, positioned on the second portion 126 of the liquid impermeable layer 114 can allow for a garment attachment mechanism located on the first portion 124 of the liquid impermeable layer 114 to dis-engage. Similarly, a release component positioned on the first portion 124 of the liquid impermeable layer 114 can allow for a garment attachment mechanism located on the second portion 126 of the liquid impermeable layer 114 to dis-engage. For example, in various embodiments, the first portion 124 of the liquid impermeable layer 114 can be provided with a garment attachment mechanism in the form of a garment attachment adhesive and the second portion 126 of the liquid impermeable layer 114 can be provided with a garment attachment mechanism in the form of a garment attachment adhesive. In such embodiments, each of the first portion 124 of the liquid impermeable layer 114 and the second portion 126 of the liquid impermeable layer 114 can also be at least partially coated with a silicone treatment as the corresponding release component. In such embodiments, the silicone treatment on the first portion 124 of the liquid impermeable layer 114 can align in a face-to-face relationship with a garment attachment adhesive on the second portion 126 of the liquid impermeable layer 114 and a silicone treatment on the second portion 126 of the liquid impermeable layer 114 can align in a face-to-face relationship with a garment attachment adhesive on the first portion 124 of the liquid impermeable layer 114. Such alignment can prevent a permanent attachment between the two portions, first portion 124 and second portion 126, of the liquid impermeable layer 114.

In various embodiments, the first region garment attachment mechanism 138 and the corresponding first region release component 140 can be provided in a pattern arrangement. In various embodiments, the first region garment attachment mechanism 138 can be provided in any pattern as deemed suitable. Exemplary patterns can include, but are not limited to, lines, swirls, dots, circles, ovals, and diamonds. In such embodiments, the corresponding first region release component 140 can be provided in a pattern arrangement which can mirror the pattern of the first region garment attachment mechanism 138.

In various embodiments, the absorbent article 10 can be provided with a maintenance component 144 which can maintain the extendable portion 102 in close proximity to the main body of the absorbent article 10 until the wearer desires to utilize the extendable portion 102. For example, with reference to FIGS. 6C and 7C, the sealed peripheral region 24 of the absorbent article 10 can comprise a maintenance component 144. The maintenance component 144 can face the extendable portion 102 and can engage with the extendable portion 102 to maintain the extendable portion 102 in close proximity to the main body of the absorbent article 10 until use by the wearer of the absorbent article 10. The maintenance component 144 can be any operative, conventional mechanism, examples of which can include, but are not limited to, adhesives, cohesives, inter-engaging mechanical fastener systems or the like, as well as combinations thereof.

To assist the wearer with extending the extendable portion 102, the extendable portion 102 can be provided with a tab 146. The tab 146 can be an integral unitary construction with the extendable portion 102 or the tab 146 can be a separate element bonded to the absorbent article 10. The tab 146 can be formed from materials similar to the topsheet layer 20, the backsheet layer 22 or combinations of these materials. The tab 146 can have any design, shape, and/or dimension deemed suitable to enable grasping of the tab 146 by the wearer to extend the extendable portion 102. The tab 146 can be grasped by the wearer of the absorbent article 10 and can be pulled in the direction indicated by the arrow (illustrated in FIGS. 6B and 7B) to extend the extendable portion 102 and create an extended region 100 for additional coverage.

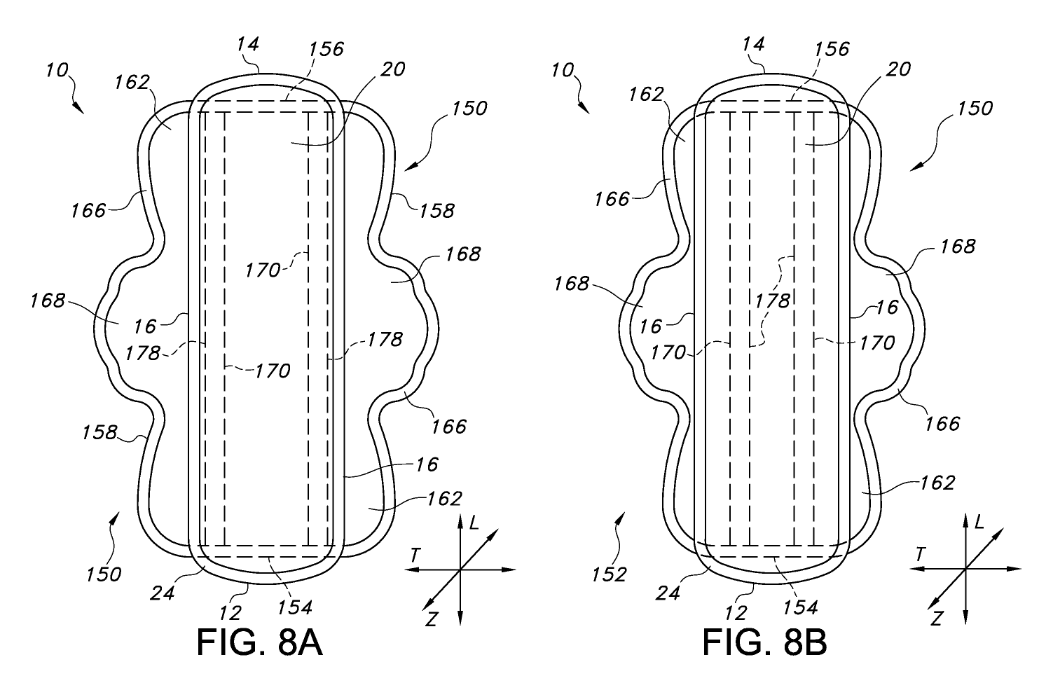

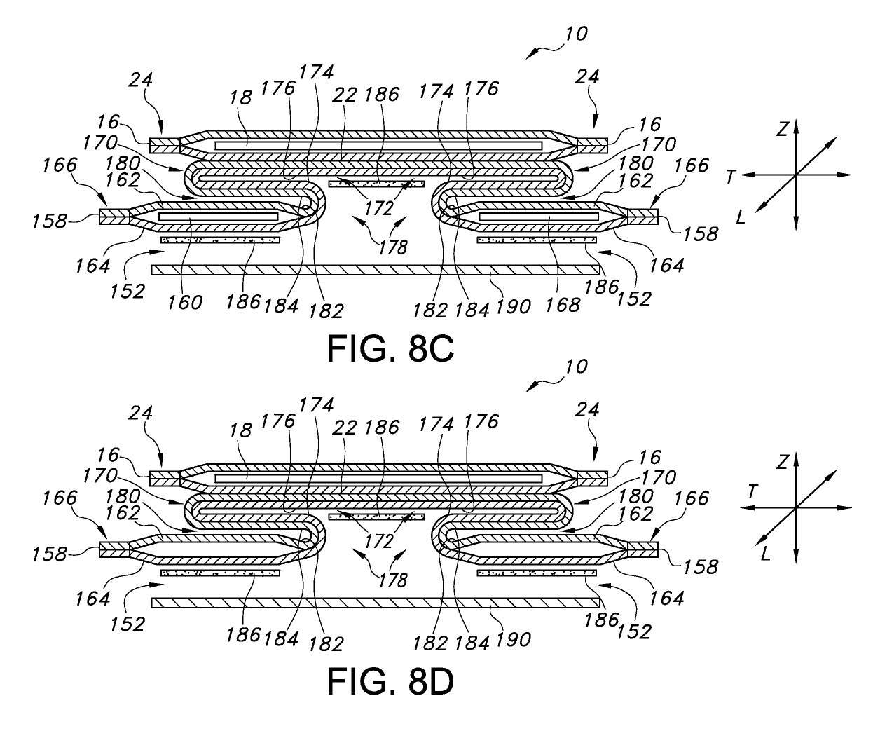

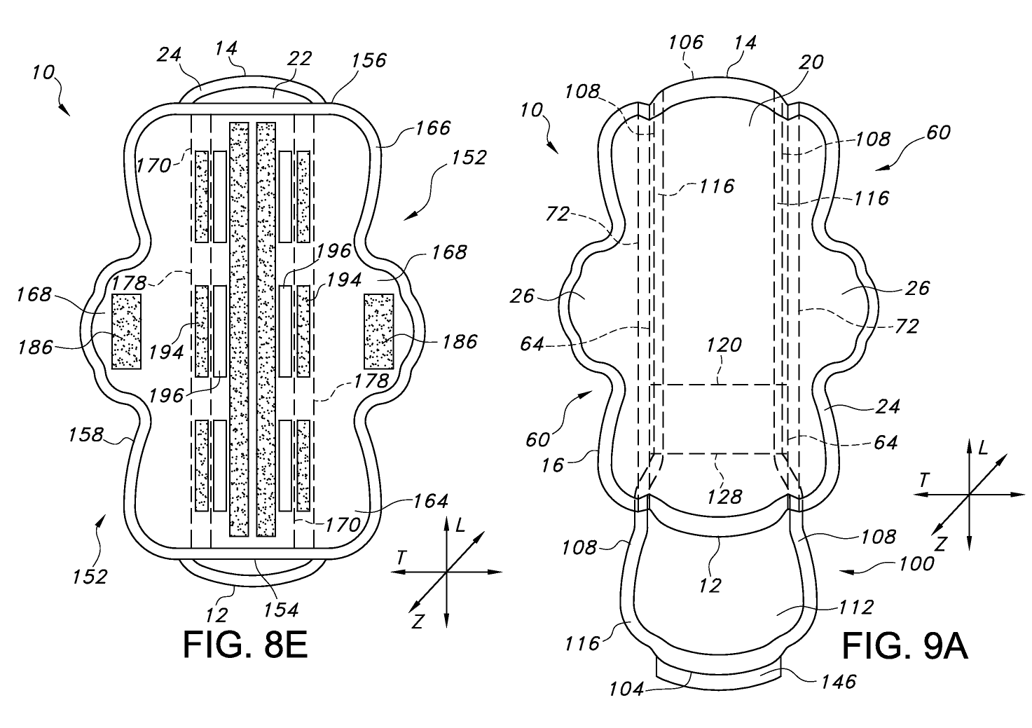

FIGS. 8A-8E provide illustrations of various views of an exemplary embodiment of an extendable absorbent article 10 having two extendable portions 152 which can extend the absorbent article 10 in the transverse direction of the absorbent article 10 and provide the absorbent article 10 with extended regions 150. In the embodiment illustrated in FIGS. 8A-8E, the extendable portions 152 are illustrated as being constructed as a laminate of at least a liquid permeable layer 162 and a liquid impermeable layer 164. FIG. 8C provides an illustration wherein the extendable portions 152 are constructed as a laminate of a liquid permeable layer 162, a liquid impermeable layer 164 and an absorbent material 160. FIG. 8D provides an illustration wherein the extendable portions 152 do not contain an absorbent material 160 within their construction. In the exemplary embodiment of the absorbent article 10 illustrated in FIGS. 8A-8E, the two extendable portions 152 are mirror images of each other and are each capable of extending the absorbent article 10 in a transverse direction of the absorbent article 10. As the two extendable portions 152 are mirror images of each other, they can extend the absorbent article 10 in opposing transverse directions of the absorbent article 10.

The absorbent article 10 can have a topsheet layer 20, a backsheet layer 22, an absorbent core 18, a pair of opposing transverse direction end edges, 12 and 14, a pair of opposing longitudinal direction side edges 16, and a sealed peripheral region 24 formed by bonding the peripheral edges of the topsheet layer 20 and the backsheet layer 22 together. The absorbent article 10 can have two extendable portions 152 constructed from a common laminate of a liquid permeable layer 162, a liquid impermeable layer 264, and in various embodiments an absorbent material 160. The laminate can further have a pair of opposing transverse direction end edges, 154 and 156, a pair of opposing longitudinal direction side edges 158, and a sealed peripheral region 166. A portion of the laminate can be permanently attached to a layer of the absorbent article 10, such as, for example the backsheet layer 22 of the absorbent article 10. In such an exemplary embodiment, the remaining portions of the laminate can remain free from permanent attachment in order to form the extendable portions 152.

Prior to usage of the absorbent article 10 by a wearer, the absorbent article 10 can be in a first configuration, such as illustrated in FIGS. 8B-8D, and can have extendable portions 152 ready for use by a wearer to extend the absorbent article 10 if so desired. The two extendable portions 152, such as illustrated in FIGS. 8B-8D, are mirror images of each other and are each independently capable of extending the absorbent article 10 in the transverse direction of the absorbent article 10, thereby increasing the first configuration first transverse width of the absorbent article 10 to a second configuration second transverse width.

As described herein, the extendable portions 152 can be incorporated into the absorbent article 10 by forming the laminate which will ultimately become the extendable portion 152 and a step of permanently attaching the laminate to a layer of an absorbent article 10, such as, for example, the backsheet layer 22 of the absorbent article 10. To form the laminate, the materials utilized for the liquid permeable layer 162 and the liquid impermeable layer 164 (and absorbent material 160 if desired) of the laminate can be sized to have the fully extended size dimensions desired for a fully extended absorbent article 10. To form the laminate the liquid permeable layer 162 and the liquid impermeable layer 164 can be bonded together at their periphery, either entirely or partially, using known bonding techniques to form a sealed peripheral region. The laminate can have, therefore, a liquid permeable layer 162, a liquid impermeable layer 164, an opposing pair of transverse direction end edges, 154 and 156, and an opposing pair of longitudinal direction side edges 158. If desired, the laminate can also have an absorbent material 160 positioned between the liquid permeable layer 162 and the liquid impermeable layer 164. At least a portion of the laminate can be permanently attached to a layer of the absorbent article 10, such as, for example, the backsheet layer 22 of the absorbent article 10 and a portion of the laminate can remain free from permanent attachment and available to be further formed into an extendable portion 152 of the absorbent article 10. In various embodiments, when the laminate is permanently attached to a layer of the absorbent article 10, the laminate can be in an unfolded configuration and at least a portion of the laminate can extend in the fully extended configuration beyond at least one of the edges, such as the longitudinal direction side edges 16, of the absorbent article 10. The portion of the laminate extending beyond at least one of the edges, 16, of the absorbent article 10 can be an extended region 150 of the absorbent article 10. A portion of the fully extended laminate can then be folded back and under the remainder of the absorbent article 10.

The portion of the fully extended laminate which can be folded can be the portion of the fully extended laminate forming the extended region 150. Incorporating a first fold into an extended region 150 can bring a portion of the extended region 150 back and under a remaining portion of the absorbent article 10. The folding of the portion of the laminate can initiate the placement of the laminate and the absorbent article 10 into a first configuration. The folded portion of the laminate can form an extendable portion 152 which can later be utilized by a wearer of the absorbent article as desired based upon the wearer's needs. The wearer can manipulate an extendable portion 152 (e.g. unfolding the extendable portion 152) to extend the dimensions of the absorbent article 10 as desired to meet their needs. If an extendable portion 152 is not needed by the wearer during use, the extendable portion 152 can remain in the folded, first configuration.

FIG. 8A provides an exemplary illustration of a potential location where a first fold, such as fold 170, can be incorporated into the extended regions 150 of the laminate. The laminate of FIGS. 8A-8E can be capable of extending the absorbent article 10 in the transverse direction of the absorbent article 10 and, therefore, the fold, such as fold 170, can extend from the first transverse direction end edge 154 to the second transverse direction end edge 156 of the laminate. As a result of incorporating each first fold 170 into the absorbent article 10, an opposing pair of first regions 172 (illustrated in FIGS. 8C and 8D) can be formed wherein, for each extendable portion 152, a first portion 174 of the liquid impermeable layer 164 can be brought into a face-to-face relationship with a second portion 176 of the liquid impermeable layer 164.

The incorporation of each of the first folds 170 into the laminate can re-position each of the longitudinal direction side edges to be closer in proximity to the opposite longitudinal direction side edge. To provide the absorbent article 10 with an extendable portion 152 a second fold 178 can be provided in order to re-direct a portion of the extendable portion 152 such that a portion of each of the extendable portions 152 extends beyond each of the first folds 170, respectively. As a result of incorporating the second folds 178 in the absorbent article 10, an opposing pair of second regions 180 can be formed wherein, for each extendable portion 152, a first portion 182 of the liquid permeable layer 162 can be brought into a face-to-face relationship with a second portion 184 of the liquid permeable layer 162 (such as seen in FIGS. 8C and 8D). The two folds, 170 and 178, can create a Z-shaped or S-shaped fold pattern in each of the extendable portions 152.

As illustrated in FIGS. 8C-8E, the liquid impermeable layer 164 of the laminate can be provided with a garment attachment mechanism 186. The garment attachment mechanism 186 can be any operative, conventional attachment mechanism such as described with regard to the absorbent article 10 of FIGS. 1A-1D. While a pattern of lines of garment attachment mechanism 186 is illustrated in FIG. 8E, the garment attachment mechanism 186 can be provided on the liquid impermeable layer 164 in any location and in any amount as deemed suitable to enable attachment between the absorbent article 10 and the wearer's undergarment. In various embodiments, the absorbent article 10 can utilize a peel strip layer 192 over a garment attachment mechanism 186 to protect the garment attachment mechanism 186 prior to usage of the absorbent article 10.

In various embodiments, while the liquid impermeable layer 164 of the laminate can be provided with a garment attachment mechanism 186, each of the first portions 174 of the liquid impermeable layer 164 and each of the second portions 176 of the liquid impermeable layer 164 which form each of the first regions 172 in the first configuration of the absorbent article 10 can each remain free of a garment attachment mechanism. In various embodiments, in each first region 172 a portion of at least one of the first portion 174 of the liquid impermeable layer 164 or the second portion 176 of the liquid impermeable layer 164 can be provided with a first region garment attachment mechanism 188. To provide for engagement between each of the first portions 174 of the liquid impermeable layer 164 and the respective second portions 176 of the liquid impermeable layer 164, the first portions 174 of the liquid impermeable layer 164 can be situated over and engaged with their respective second portions 176 of the liquid impermeable layer 164 via the first region garment attachment mechanism 188 located within each of the first regions 172. Engagement between the first portions 174 of the liquid impermeable layer 164 and their respective second portions 176 of the liquid impermeable layer 164 can provide for an opposing pair of extendable portions 152 which can remain in a first configuration. When a wearer of the absorbent article 10 desires to extend the extendable portions 152, at least one of the first portions 174 of the liquid impermeable layer 164 can be dis-engaged from its respective second portion 176 of the liquid impermeable layer 164 in a conversion of the absorbent article 10 from a first configuration to a second configuration. A first region garment attachment mechanism 188 can also help secure the extended region 150 of the absorbent article 10 to a wearer's undergarment in the event that the wearer has extended the extendable portion 152 of the absorbent article 10.

In various embodiments, to provide for engagement between each of the first portions 174 of the liquid impermeable layer 164 and their respective second portions 176 of the liquid impermeable layer 164 in each of the first regions 172, a first region garment attachment mechanism 188 can be positioned on a portion of each of the first portions 174 of the liquid impermeable layer 164 and/or a portion of each of the second portions 176 of the liquid impermeable layer 164 in each of the first regions 172. The first portions 174 of the liquid impermeable layer 174 can then be brought into contact with their respective second portions 176 of the liquid impermeable layer 164 and the first region garment attachment mechanisms 188 can maintain engagement between the first portions 174 and the second portions 176 of the liquid impermeable layer 164. In various embodiments in which the first portions 174 of the liquid impermeable layer 164 are engaged with a second portions 176 of the liquid impermeable layer 164 via a first region garment attachment mechanism 188, it can be beneficial to dis-engage a first portion 174 of the liquid impermeable layer from a second portion 76 of the liquid impermeable layer 164 in order to allow for extension of an individual extendable portion 152 as desired by the wearer of the absorbent article 10. To provide for such releasable engagement between one of the first portions 174 of the liquid impermeable layer 164 and its respective second portion 176 of the liquid impermeable layer 164, a portion of the first portion 174 of the liquid impermeable layer 164 and/or a portion of the second portion 176 of the liquid impermeable layer 164 which would be positioned opposite the first region garment attachment mechanism 188 can be provided with a corresponding first region release component 190.

For example, in various embodiments, at least a portion of a first portion 174 of the liquid impermeable layer 164 can be provided with a first region garment attachment mechanism 188 in the form of a garment attachment adhesive and at least a portion of a second portion 176 of the liquid impermeable layer 164 can be coated with a silicone treatment, as the first region release component 190, in a location of the second portion 176 of the liquid impermeable layer 164 which will be opposite the first region garment attachment mechanism 188 positioned on the first portion 174 of the liquid impermeable layer 164. When the first portion 174 of the liquid impermeable layer 164 and its respective second portion 176 of the liquid impermeable layer 164 are in a first configuration such as illustrated in FIGS. 8B-8D, the first region release component 190, such as the silicone treatment, is in a face-to-face relationship with the first region garment attachment mechanism 188, such as the garment attachment adhesive, positioned on the first portion 174 of the liquid impermeable layer 164. In such embodiments, the silicone treatment which is in the face-to-face relationship with the garment attachment adhesive can prevent a permanent engagement between the first portion 174 of the liquid impermeable layer 164 and the second portion 176 of the liquid impermeable layer 164. The first portion 164 of the liquid impermeable layer 164 can, therefore, be capable of dis-engaging from the second portion 176 of the liquid impermeable layer 164 when the wearer desires to extend the extendable portion 152.

Referring to FIG. 8E, a bottom view of an exemplary absorbent article 10 extended in a fully extended configuration is illustrated. FIG. 8E provides an illustration of potential locations where folds, such as folds 170 and 178, can be incorporated into the laminate. In various embodiments, the fully extended configuration of an absorbent article 10 can exist such as, for example, during the manufacturing process prior to incorporating folds, such as folds 170 and 178, into the absorbent article 10. In various embodiments, the fully extended configuration can exist, such as, for example, during usage when a wearer has altered the configuration of the absorbent article 10 from the first configuration to a second configuration by extending the extendable portion 152 into an extended region 150.

As illustrated in FIG. 8E, a portion of the liquid impermeable layer 164 which will form the first portions 174 of the liquid impermeable layer 164 in the first region 172 when the absorbent article 10 is in a first configuration can be provided with a first region garment attachment mechanism 188 and a portion of the liquid impermeable layer 164 which will form the second portions 176 of the liquid impermeable layer 164 in the first region 172 when the absorbent article 10 is in a first configuration can be provided with a corresponding first region release component 190. When the absorbent article 10 is in a first configuration, the first region garment attachment mechanism 188 and the first region release component 190 can align with each other and can be in a face-to-face relationship. Such alignment can prevent a permanent attachment between the first portions 174 and the second portions 176 of the liquid impermeable layer 164. While the first portions 174 have been illustrated with a first region garment attachment mechanism 188 and the second portions 176 have been illustrated with a first region release component 190, it is to be understood that each of the first region garment attachment mechanism 188 and the first region release component 190 can be located on either or both of the first portions 174 and/or second portion 176 of the liquid impermeable layer 164 in the first regions 172.

In various embodiments, each of the first portions 174 and the second portions 176 of the liquid impermeable layer 164 can be provided with a garment attachment mechanism. In such embodiments in which both the first portions 174 and the second portions 176 of the liquid impermeable layer 164 have a garment attachment mechanism, each of the first portions 174 and second portions 176 of the liquid impermeable layer 164 can have a corresponding release component, positioned opposite a garment attachment mechanism on the opposing portion of the liquid impermeable layer 164, which can allow for the garment attachment mechanisms to dis-engage and allow for the unfolding of the extendable portions 152. Therefore, a release component, positioned on the second portions 176 of the liquid impermeable layer 164 can allow for a garment attachment mechanism located on the first portions 174 of the liquid impermeable layer 164 to dis-engage. Similarly, a release component positioned on the first portions 174 of the liquid impermeable layer 164 can allow for a garment attachment mechanism located on the second portions 176 of the liquid impermeable layer 164 to dis-engage. For example, in various embodiments, the first portions 174 of the liquid impermeable layer 164 can be provided with a garment attachment mechanism in the form of a garment attachment adhesive and the second portions 176 of the liquid impermeable layer 164 can be provided with a garment attachment mechanism in the form of a garment attachment adhesive. In such embodiments, each of the first portions 174 of the liquid impermeable layer and the second portions 176 of the liquid impermeable layer 164 can also be at least partially coated with a silicone treatment as the corresponding release component. In such embodiments, the silicone treatment on the first portions 174 of the liquid impermeable layer 164 can align in a face-to-face relationship with a garment attachment adhesive on the second portions 176 of the liquid impermeable layer 164 and a silicone treatment on the second portions 176 of the liquid impermeable layer 164 can align in a face-to-face relationship with a garment attachment adhesive on the first portions 174 of the liquid impermeable layer 164. Such alignment can prevent a permanent attachment between the two portions, first portions 174 and second portions 176, of the liquid impermeable layer 164.

In various embodiments, the first region garment attachment mechanism 188 and the corresponding first region release component 190 can be provided in a pattern arrangement. In various embodiments, the first region garment attachment mechanism 188 can be provided in any pattern as deemed suitable. Exemplary patterns can include, but are not limited to, lines, swirls, dots, circles, ovals, and diamonds. In such embodiments, the corresponding first region release component 190 can be provided in a pattern arrangement which can mirror the pattern of the first region garment attachment mechanism 188.

The laminate can be provided with wings 168. The wings 168 can provide a location for a wearer of the absorbent article 10 to grab the extendable portions 152 and pull the extendable portions 152 in the direction indicated by the arrows (in FIG. 8B) in order to create extended regions 150 of the absorbent article 10. In the event that the absorbent article 10 is not provided with wings 168, to assist the wearer with extending the extendable portions 152, the extendable portions can be provided with a tab. The tab can be an integral unitary construction with the extendable portions 152 or the tab can be a separate element bonded to the absorbent article 10. The tab can be formed from materials similar to the topsheet layer 20, the backsheet layer 22 or combinations of these materials. The tab can have any design, shape, and/or dimension deemed suitable to enable grasping of the tab by the wearer to extend the extendable portions 152. The tab can be grasped by the wearer of the absorbent article 10 and can be pulled in the direction indicated by the arrow to extend the extendable portions 152 and create an extended region 150 for additional coverage.

FIGS. 9A-9C provide illustrations of various views of an exemplary embodiment of an extendable absorbent article 10 having one extendable portion 102, which is capable of extending the absorbent article 10 in the longitudinal direction of the absorbent article 10, and two extendable portions 62, which are capable of extending the absorbent article 10 in the transverse direction of the absorbent article 10. FIG. 9A provides a top view illustration of an exemplary embodiment of an extendable absorbent article 10 in a fully extended configuration wherein the absorbent article 10 has an extended region 100 in the longitudinal direction of the absorbent article 10 and a pair of extended regions 60 in the transverse direction of the absorbent article 10. As can be seen from FIGS. 9A-9C, the absorbent article 10 of FIGS. 9A-9C can be a combination of the absorbent article 10 of FIGS. 6A-6D and the absorbent article 10 of FIGS. 3A-3E.

In various embodiments, the longitudinal direction extendable portion 102 can be constructed as a laminate of at least a liquid permeable layer 112 and a liquid impermeable layer 114 (and in various embodiments an absorbent material, such as absorbent material 110 of FIGS. 6A-6D). In various embodiments, the transverse direction extendable portions 62 can be constructed as extensions of the topsheet layer 20 and the backsheet layer 22 of the absorbent article 10. In various embodiments, the transverse direction extendable portions 62 can be constructed as extensions of the topsheet layer 20, the backsheet layer 22, and an absorbent material.

In various embodiments, in the first configuration of the absorbent article 10, the extendable portion 102 can overlay the pair of extendable portions 62. For example, in the first configuration, at least a portion of each of the extendable portions 62 can be positioned in a portion of the first region 122 of the absorbent article 10. In various embodiments, in the first configuration of the absorbent article 10, the extendable portions 62 can overlay the extendable portion 102. For example, in the first configuration, at least a portion of the extendable portion 102 can be positioned within a portion of the first region 66 of each of the extendable portions 62. In various embodiments, in the first configuration of the absorbent article 10, the absorbent article 10 can be sized such that there is no overlap between the extendable portion 102 and the pair of extendable portions 62.

FIGS. 10A-10C provide illustrations of various views of an exemplary embodiment of an extendable absorbent article 10 having two extendable portions 102, which are capable of extending the absorbent article 10 in the longitudinal direction of the absorbent article 10, and two extendable portions 62, which are capable of extending the absorbent article 10 in the transverse direction of the absorbent article 10. FIG. 10A provides a top view illustration of an exemplary embodiment of an extendable absorbent article 10 in a fully extended configuration wherein the absorbent article 10 has two extended regions 100 in the longitudinal direction of the absorbent article 10 and a pair of extended regions 60 in the transverse direction of the absorbent article 10. As can be seen from FIGS. 10A-10C, the absorbent article 10 of FIGS. 10A-10C can be a combination of the absorbent article 10 of FIGS. 7A-7D and the absorbent article 10 of FIGS. 3A-3E.

In various embodiments, the longitudinal direction extendable portions 102 can be constructed as laminates of a liquid permeable layer 112 and a liquid impermeable layer 114 (and in various embodiments an absorbent material such as absorbent material 110 of FIGS. 7A-7D). In various embodiments, the transverse direction extendable portions 62 can be constructed as extensions of the topsheet layer 20 and the backsheet layer 22 of the absorbent article 10. In various embodiments, the transverse direction extendable portions 62 can be constructed as extensions of the topsheet layer 20, the backsheet layer 22, and an absorbent material.

In various embodiments, in the first configuration of the absorbent article 10, the pair of extendable portions 102 can overlay the pair of extendable portions 62. For example, in the first configuration, at least a portion of each of the extendable portions 62 can be positioned in a portion of each of the first regions 122 of the absorbent article 10. In various embodiments, in the first configuration of the absorbent article 10, the extendable portions 62 can overlay the extendable portions 102. For example, in the first configuration, at least a portion of the extendable portions 102 can be positioned within a portion of the first region 66 of each of the extendable portions 62. In various embodiments, in the first configuration of the absorbent article 10, the absorbent article 10 can be sized such that there is no overlap between the extendable portions 102 and the pair of extendable portions 62.

In various embodiments, an absorbent article 10 can have three extendable portions in which one extendable portion is capable of extending the absorbent article 10 in the longitudinal direction and two extendable portions are capable of extending the absorbent article 10 in the transverse direction. In such embodiments, each of the extendable portions can be constructed as laminates of a liquid permeable layer, a liquid impermeable layer, and in various embodiments an absorbent material. In such embodiments, the absorbent article 10 can be a combination of the absorbent article 10 of FIGS. 6A-6D and the absorbent article 10 of FIGS. 8A-8E.

In various embodiments, an absorbent article 10 can have four extendable portions in which two extendable portions are capable of extending the absorbent article 10 in the longitudinal direction and two extendable portions are capable of extending the absorbent article 10 in the transverse direction. In such embodiments, each of the extendable portions can be constructed as laminates of a liquid permeable layer, a liquid impermeable layer and in various embodiments an absorbent material. In such embodiments, the absorbent article 10 can be a combination of the absorbent article 10 of FIGS. 7A-7D and the absorbent article 10 of FIGS. 8A-8E.

Transfer Delay Layer:

In various embodiments, the absorbent article 10 can include a liquid permeable transfer delay layer (not shown) positioned below the topsheet layer 20 in the depth (Z) direction. The transfer delay layer may contain a material that is substantially hydrophobic. For example, the transfer delay layer may be a nonwoven fibrous web composed of relatively hydrophobic materials, such as polypropylene, polyethylene, polyester, or the like, and also may be composed of a blend of such materials. One example of a material suitable for the transfer delay layer can be a spunbond web composed of polypropylene, multi-lobal fibers. Further examples of suitable transfer delay layers can include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay layer are described in U.S. Pat. No. 4,798,603 to Meyer, et al. and U.S. Pat. No. 5,248,309 to Serbiak, et al.

The transfer delay layer may generally have any size, such as a length of about 150 mm to about 300 mm. Typically, the length of the transfer delay layer can be approximately equal to the length of the absorbent article 10. The width of the transfer delay layer can be from between about 50 mm to about 75 mm. The transfer delay layer can have a basis weight less than about 250 gsm, and in some embodiments, between about 40 gsm and about 200 gsm.

Fluid Intake Layer:

In various embodiments, the absorbent article 10 can include a liquid permeable fluid intake layer (not shown) positioned between the topsheet layer 20 and the absorbent core 18. Such an intake layer can be made of a material that can be capable of rapidly transferring, in the Z-direction, body exudates that are delivered to the topsheet layer 20. The intake layer can generally have any shape and/or size desired. In an embodiment, the intake layer can have a curved rectangular shape, with a length equal to or less than the overall length of the absorbent article 10, and a width less than the width of the absorbent article 10. For example, a length of between about 150 mm to about 300 mm and a width of between about 10 mm to about 60 mm may be utilized. Any of a variety of different materials can be capable of being used for the intake layer to accomplish the above-mentioned functions. The material may be synthetic, cellulosic, or a combination of synthetic and cellulosic materials. For example, airlaid cellulosic tissues may be suitable for use in the intake layer. The airlaid cellulosic tissue may have a basis weight ranging from about 10 gsm to about 300 gsm, and in some embodiments, between about 100 gsm to about 250 gsm. The airlaid cellulosic tissue can be formed from hardwood and/or softwood fibers. An airlaid cellulosic tissue can have a fine pore structure and can provide an excellent wicking capacity, especially for menses.

Additionally, to further enhance the ability of the absorbent article 10 to transfer body exudates in the depth (Z) direction from the topsheet layer 20 toward any lower layers in the absorbent article 10 as well as to enhance the ability of the intake layer to conform to the wearer's body based on its ability to bend, the intake layer can have an opening in the layer which can be any suitable shape, such as ovular, circular, rectangular, square, triangular, etc. The opening in the intake layer can serve to funnel and direct body exudates from the topsheet layer 20 and towards lower layers of the absorbent article 10 in the depth (Z) direction. The opening can also form a cup or well-like structure for holding fluid and preventing its leakage away from a central region of the absorbent article 10 and towards the edges of the absorbent article 10.

Additional Layers:

Additional layers between the topsheet layer 20 and the absorbent core 18 can include surge layers as are commonly known. Surge layers (not shown) can be constructed of any woven or nonwoven material that is easily penetrated by body exudates. The surge layers can help to absorb, decelerate, and diffuse surges or gushes of liquid that may be rapidly introduced into the absorbent article 10. The surge layers can rapidly accept and temporarily hold the liquid prior to releasing the liquid into, for instance, the absorbent core 18 or any other layer of the absorbent article 10. Various woven fabrics and nonwoven webs can be used to construct the surge layers. For example, the surge layers can comprise a nonwoven fabric layer composed of a meltblown or spunbond web of polyolefin or polyester filaments. Such nonwoven fabric layers may include conjugate, biconstituent and homopolymer fibers of staple or other lengths and mixtures of such fibers with other types of fibers. The surge layers can also be a bonded card web or an airlaid web composed of natural and/or synthetic fibers. The bonded carded web may, for example, be a powder bonded carded web, an infrared bonded carded web, or a through air bonded carded web. The bonded carded webs can optionally include a mixture or blend of different fibers. The surge layers typically have a basis weight of less than about 100 gsm, and in some embodiments, from about 10 gsm to about 40 gsm.

Still another layer that may be present between the topsheet layer 20 and the absorbent core 18 can include a bicomponent fluid distribution layer (not shown), which can increase absorbency by providing a high void space and may be made of a through air bonded carded web, having a basis weight, in an embodiment, of between about 25 gsm and 100 gsm.

Wings:

The wings 26 can be constructed from materials described above with respect to the topsheet layer 20 and the backsheet layer 22. In various embodiments, the wings 26 can comprise an extension of a layer of material within the topsheet layer 20 and/or the backsheet layer 22. By way of example, the wings 26 can be formed by an extension of the topsheet layer 20 and backsheet layer 22 that are then bonded together along peripheral seal 24. Such wings 26 can be integrally formed with the main portion of the absorbent article 10. Alternatively, the wings 26 can be formed independently and separately attached to an intermediate section of the absorbent article 10. Wings 26 that are made independent of the other components of the absorbent article 10 can be bonded to a portion of the topsheet layer 20 and/or backsheet layer 22. Examples of processes for manufacturing absorbent articles 10 and wings 26 include, but are not limited to, those described in U.S. Pat. No. 4,059,114 to Richards, U.S. Pat. No. 4,862,574 to Hassim, et al., U.S. Pat. No. 5,342,647 to Heindel, et al., U.S. Pat. No. 7,070,672 to Alcantara, et al., U.S. Publication No. 2004/0040650 to Venturino, et al., and international publication WO1997/040804 to Emenaker, et al., each of which are hereby incorporated by reference thereto in its entirety.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article characterized by comprising:
a. a longitudinal direction and a transverse direction;
b. a first longitudinal length and a first transverse width;
c. a topsheet layer, a backsheet layer, and an absorbent core positioned between the topsheet layer and the backsheet layer;
d. a first extendable portion comprising a first portion of the topsheet layer, a first portion of the backsheet layer and a folded configuration;
e. a second extendable portion comprising a second portion of the topsheet layer, a second portion of the backsheet layer and a folded configuration; and
f. a first configuration capable of converting to a second configuration, wherein the first extendable portion is capable of extending the absorbent article in the longitudinal direction in the conversion of the absorbent article from the first configuration to the second configuration, and wherein the second extendable portion is capable of extending the absorbent article in the transverse direction of the absorbent article.

2. The absorbent article of claim 1 wherein the first extendable portion further comprises a portion of the absorbent core between the topsheet layer and the backsheet layer.

3. The absorbent article of claim 1 wherein the second extendable portion further comprises a portion of the absorbent core between the topsheet layer and the backsheet layer.

4. The absorbent article of claim 1 wherein the folded configuration of the first extendable portion further comprises a garment attachment mechanism.

5. The absorbent article of claim 1 wherein the folded configuration of the second extendable portion further comprises a garment attachment mechanism.

6. The absorbent article of claim 1 wherein the folded region of the first extendable portion is one of a Z-fold or S-fold.

7. The absorbent article of claim 1 wherein the folded region of the second extendable portion is one of a Z-fold or S-fold.

8. An absorbent article comprising:
a. a longitudinal direction and a transverse direction;
b. a first longitudinal length and a first transverse width;
c. a topsheet layer, a backsheet layer, and an absorbent core positioned between the topsheet layer and the backsheet layer;
d. a first extendable portion comprising a laminate of a liquid permeable layer, a liquid impermeable layer and a folded configuration, wherein at least a portion of the laminate is permanently attached to the backsheet layer; and
e. a first configuration capable of converting to a second configuration, wherein the first extendable portion is capable of extending the absorbent article in one of the longitudinal direction or transverse direction in the conversion of the absorbent article from the first configuration to the second configuration.

9. The absorbent article of claim 8 wherein a portion of the liquid permeable layer of the first extendable portion is in a face-to-face relationship with the backsheet layer.

10. The absorbent article of claim 8 wherein the first extendable portion further comprises an absorbent material positioned between the liquid permeable layer and the liquid impermeable layer.

11. The absorbent article of claim 8 further comprising a second extendable portion comprising a laminate of a liquid permeable layer, a liquid impermeable layer, and a folded configuration, wherein at least a portion of the laminate is permanently attached to the backsheet layer, and wherein the second extendable portion is capable of extending the absorbent article in one of the longitudinal direction or the transverse direction of the absorbent article.

12. The absorbent article of claim 11 wherein the second extendable portion further comprises an absorbent material positioned between the liquid permeable layer and the liquid impermeable layer.

13. The absorbent article of claim 8 further comprising a second extendable portion comprising a first portion of the topsheet layer, a first portion of the backsheet layer and a folded configuration, wherein the second extendable portion is capable of extending the absorbent article in one of the longitudinal direction or the transverse direction of the absorbent article.

14. The absorbent article of claim 13 wherein the second extendable portion further comprises a portion of the absorbent core between the topsheet layer and the backsheet layer.

15. The absorbent article of claim 11 wherein the first extendable portion is capable of extending in the longitudinal direction and the second extendable portion is capable of extending in the transverse direction.

16. The absorbent article of claim 11 wherein the first extendable portion is capable of extending in the transverse direction and the second extendable portion is capable of extending in the longitudinal direction.
